(19) United States
(12) Patent Application Publication
(10) Pub. No.: US 2012/0004235 A1
(43) Pub. Date: Jan. 5, 2012

(54) METHODS TO TREAT CANCER

(75) Inventors: Edmond J. Lavoie, New Brunswick, NJ (US); Beverly Teicher, Cambridge, MA (US); Steven Schmid, San Antonio, TX (US)

(73) Assignees: Rutgers, The State University of New Jersey, New Brunswick, NJ (US); Genzyme Corporation, Cambridge, MA (US); Old Queens Building, Somerset and George Streets, Brunswick, NJ (US)

(21) Appl. No.: 13/146,555

(22) PCT Filed: Jan. 29, 2010
(86) PCT No.: PCT/US10/22625

§ 371 Date: Jul. 27, 2011

Related U.S. Application Data
(60) Provisional application No. 61/148,881, filed on Jan. 30, 2009.
(60) Provisional application No. 61/240,873, filed on Sep. 9, 2009.

(51) Int. Cl. A61K 31/5025; A61P 35/00; A61K 31/4375

(52) U.S. Cl. 514/248; 514/280

(57) ABSTRACT

The invention provides methods and pharmaceutical compositions for treating certain cancers with compounds of formula (I) wherein A, B, W, Y, Z, and R₁ have any of the meanings defined in the specification and their pharmaceutically acceptable salts and prodrugs.

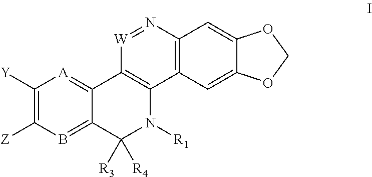

This application claims priority from U.S. Provisional Application No. 61/148,881 filed on 30 Jan. 2009 and from U.S. Provisional Application No. 61/240,873 filed on 9 Sep. 2009. The entire content of each of these provisional applications is hereby incorporated herein by reference.

DNA-topoisomerases are enzymes which are present in the nuclei of cells where they catalyze the breaking and rejoining of DNA strands, which control the topological state of DNA. Recent studies also suggest that topoisomerases are also involved in regulating template supercoiling during RNA transcription. There are two major classes of mammalian topoisomerases. DNA-topoisomerase-I catalyzes changes in the topological state of duplex DNA by performing transient single-strand breakage-union cycles. In contrast, mammalian topoisomerase II alters the topology of DNA by causing a transient enzyme bridged double-strand break, followed by strand passing and resealing. Mammalian topoisomerase II has been further classified as Type IIα. and Type II β. The antitumor activity associated with agents which are topoisomerase poisons is associated with their ability to stabilize the enzyme-DNA cleavable complex. This drug-induced stabilization of the enzyme-DNA cleavable complex effectively converts the enzyme into a cellular poison.

Several antitumor agents in clinical use have potent activity as mammalian topoisomerase II poisons. These include adriamycin, actinomycin D, daunomycin, VP-16, and VM-26 (teniposide or epipodophyllotoxin). In contrast to the number of clinical and experimental drugs which act as topoisomerase II poisons, there are currently only a limited number of agents which have been identified as topoisomerase I poisons. Camptothecin and its structurally-related analogs are among the most extensively studied topoisomerase I poisons. Bi- and terbenzimidazoles (Chen et al., Cancer Res. 1993, 53, 1332-1335; Sun et al., J. Med. Chem. 1995, 38, 3638-3644; Kim et al., J. Med. Chem. 1996, 39, 992-998), certain benzo[c]phenanthridine and protoberberine alkaloids and their synthetic analogs (Makhey et al., Med. Chem. Res. 1995, 5, 1-12; Janin et al., J. Med. Chem. 1975, 18, 708-713; Makhey et al., Bioorg. & Med. Chem. 1996, 4, 781-791), as well as the fungal metabolites, bulgarein (Fujii et al., J. Biol. Chem. 1993, 268, 13160-13165) and saintopin (Yamashita et al., Biochemistry 1991, 30, 5838-5845) and indolocarbazoles (Yamashita et al., Biochemistry 1992, 31, 12069-12075) have been identified as topoisomerase I poisons. Other topoisomerase poisons have been identified including certain benzo[i]phenanthridine and cinnoline compounds (see LaVoie et al., U.S. Pat. No. 6,140,328, and WO 01/32631. While these compounds are useful they are somewhat limited due to low solubility.

F.D.A. approved Topoisomerase I inhibitors are camptothecin derivatives and include CAMPTOSAR® (irinotecan) and HYCAMTIN® (topotecan). CAMPTOSAR® (irinotecan) is indicated as a component of first-line therapy in combination with 5-fluorouracil and leucovorin for patients with metastatic carcinoma of the colon or rectum. CAMPTOSAR® (irinotecan)is also indicated for patients with metastatic carcinoma of the colon or rectum whose disease has recurred or progressed following initial fluorouracil-based therapy. SN-38 is a well known active metabolite of irinotecan. HYCAMTIN® (topotecan) is indicated for treatment of patients with relapsed small cell lung cancer in patients with a prior complete or partial response and who are at least 45 days from the end of first-line chemotherapy. As mentioned above, these camptothecin derivatives suffer from low solubility.

There thus is a need for non-camptothecin based Topoisomerase I inhibitors that are therapeutically effective against cancers.

International patent application number PCT/US02/36901 discusses compounds of formula I:

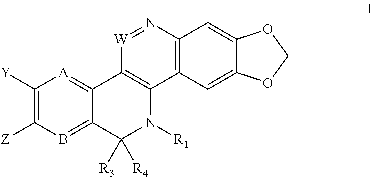

that are reported to have topoisomerase inhibiting activity. The compounds of formula I are non-camptothecin derivatives, and as such, are not burdened with certain shortcomings of camptothecin based derivatives. Applicant has discovered that compounds of formula I are particularly active against certain specific types of cancer (e.g. colon cancer, non-small cell lung cancer (NSCLC), melanoma, NCI-H292 lung cancer, renal cancer, H1299 lung cancer, colorectal cancer, cervical cancer, breast cancer, and multiple myeloma). Particularly preferred compounds include 8,9-dimethoxy-2,3-methylenedioxy-5-[2-(N,N-dimethylamino)ethyl]-5H-dibenzo[c,h]1,6-naphthyridin-6-one; 8,9-dimethoxy-2,3-methylenedioxy-5-[2-(N,N-diethylamino)ethyl]-5H-dibenzo[c,h]1,6-naphthyridin-6-one; and 8,9-dimethoxy-2,3-methylenedioxy-5-[2-(N-methylamino)ethyl]-5H-dibenzo[c,h]1,6-naphthyridin-6-one; and pharmaceutically acceptable salts and prodrugs thereof.

Accordingly, in one embodiment the invention provides a method for treating a cancer selected from colon cancer, non-small cell lung cancer (NSCLC), melanoma, NCI-H292 lung cancer, renal cancer, H1299 lung cancer, colorectal cancer, cervical cancer, breast cancer, and multiple myeloma in a mammal comprising administering to the mammal an effective amount of a compound of formula I:

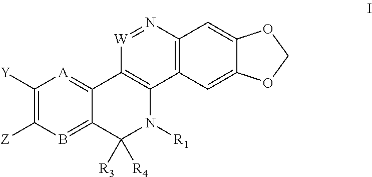

wherein:

A and B are independently N or CH;

W is N or CH;

R₃ and R₄ are each independently H, (C₁-C₆)alkyl, or substituted (C₁-C₆)alkyl, or R₃ and R₄ together are ═O, ═NH or ═N—R₂;

Y and Z are independently hydroxy, (C₁-C₆)alkoxy, substituted (C₁-C₆)alkoxy, (C₁-C₆)alkanoyloxy, substituted (C₁-C₆) alkanoyloxy, —O—P(═O)(OH)₂, or —O—C(═O)NR_{c}R_{d}; or Y and Z together with the ring carbon atoms to which they are attached form an alkylenedioxy ring with from 5 to 7 ring atoms;

R₁ is a -(C₁-C₆)alkyl substituted with one or more solubilizing groups;

R₂ is (C₁-C₆)alkyl or substituted (C₁-C₆)alkyl; and

R_{c} and R_{d} are each independently (C₁-C₆) alkyl or substituted (C₁-C₆) alkyl; or R_{c} and R_{d} together with the nitrogen to which they are attached form a N′-{(C₁-C₆)alkyl}piperazino, pyrrolidino, or piperidino ring, which ring can optionally be substituted with one or more aryl, heteroaryl, or heterocycle;

or a pharmaceutically acceptable salt or prodrug thereof.

The invention also provides a pharmaceutical composition for the treatment of cancer (e.g., colon cancer, non-small cell lung cancer (NSCLC), melanoma, NCI-H292 lung cancer, renal cancer, H1299 lung cancer, colorectal cancer, cervical cancer, breast cancer, and multiple myeloma) comprising a compound of formula I or a pharmaceutically acceptable salt or prodrug thereof and a pharmaceutically acceptable excipient. In certain embodiments, the compound of formula I is 8,9-dimethoxy-2,3-methylenedioxy-5-[2-(N,N-dimethylamino)ethyl]-5H-dibenzo[c,h]1,6-naphthyridin-6-one; 8,9-dimethoxy-2,3-methylenedioxy-5-[2-(N,N-diethylamino)ethyl]-5H-dibenzo[c,h]1,6-naphthyridin-6-one; or 8,9-dimethoxy-2,3-methylenedioxy-5-[2-(N-methylamino)ethyl]-5H-dibenzo[c,h]1,6-naphthyridin-6-one; or a pharmaceutically acceptable salt or prodrug thereof.

The invention also provides a compound of formula I or a pharmaceutically acceptable salt or prodrug thereof for use in the prophylactic or therapeutic treatment of cancer (e.g. colon cancer, non-small cell lung cancer (NSCLC), melanoma, NCI-H292 lung cancer, renal cancer, 111299 lung cancer, colorectal cancer, cervical cancer, breast cancer, and multiple myeloma).

The invention also provides the use of a compound of formula I or a pharmaceutically acceptable salt or prodrug thereof for the manufacture of a medicament useful for the treatment of cancer (e.g. colon cancer, non-small cell lung cancer (NSCLC), melanoma, NCI-H292 lung cancer, renal cancer, H1299 lung cancer, colorectal cancer, cervical cancer, breast cancer, and multiple myeloma) in a mammal.

### BRIEF DESCRIPTION OF THE FIGURES

FIG. 1 shows the mean tumor volume of mice treated with Compound 2 citrate salt vs. HCT-116.

FIG. 2 shows the mean tumor volume of mice treated with Compound 2 citrate salt (IP; QOD 3 for 2 cycles) or Docetaxel (IV; QOD 3) vs . NCI-H460.

FIG. 3 shows the mean tumor volume of mice treated with Compound 2 citrate salt (IP) or Irinotecan (IP) vs. NCI-H460

FIG. 4 shows the mean tumor volume of mice treated with Compound 2 citrate salt (IP; QODx3 for 2 cycles) or Irinotecan (IV; Q4Dx3) vs. HT-29

FIG. 5 shows the mean tumor volume of mice treated with Compound 2 citrate salt (IP) vs. Comparator Agents (IP) in NCI-H460

FIG. 6 shows the mean tumor volume of mice treated with Compound 2 citrate salt vs. Comparator Agents in MDA-MB-231 Human Breast Tumor.

FIG. 7 shows the mean tumor volume of mice treated with Compound 2 citrate salt vs. HCT-116 Human Colorectal Tumor.

### DETAILED DESCRIPTION

The following defmitions are used, unless otherwise described.

“(C₁-C₆)alkyl” denotes both straight and branched carbon chains with one or more, for example, 1, 2, 3, 4, 5, or 6, carbon atoms, but reference to an individual radical such as “propyl” embraces only the straight chain radical, a branched chain isomer such as “isopropyl” being specifically referred to.

“Substituted (C₁-C₆)alkyl” is an alkyl group of the formula (C₁-C₆)alkyl as defined above wherein one or more (e.g. 1 or 2) carbon atoms in the alkyl chain have been replaced with a heteroatom independently selected from —O—, —S— and NR— (where R is hydrogen or C₁-C₆alkyl) and/or wherein the alkyl group is substituted with from 1 to 5 substituents independently selected from cycloalkyl, substituted cycloalkyl, (C₁-C₆)alkoxycarbonyl (e.g. —CO₂Me), cyano, halo, hydroxy, oxo (═O), carboxy (COOH), aryloxy, heteroaryloxy, heterocyclooxy, nitro, and —NR^{a}R^{b}, wherein R^{a} and R^{b} may be the same or different and are chosen from hydrogen, alkyl, arylalkyl, heteroarylalkyl, heterocycloalkyl, cycloalkyl, substituted cycloalkyl, aryl, heteroaryl and heterocyclic. Substituted (C₁-C₆)alkyl groups are exemplified by, for example, groups such as hydroxymethyl, hydroxyethyl, hydroxypropyl, 2-aminoethyl, 3-aminopropyl, 2-methylaminoethyl, 3-dimethylaminopropyl, 2-carboxyethyl, hydroxylated alkyl amines, such as 2-hydroxyaminoethyl, and like groups. Specific substituted (C₁-C₆)alkyl groups are (C₁-C₆)alkyl groups substituted with one or more substituents of the formula —NRₐR_{b} where Rₐ and R_{b} together with the nitrogen to which they are attached form of nitrogen containing heterocyclic ring. Specific examples of such heterocyclic rings include piperazino, pyrrolidino, piperidino, morpholino, or thiomorpholino. Other specific substituted (C₁-C₆)alkyl groups are (C₁-C₆)alkyl groups substituted with one or more carbon-linked oxygen containing heterocyclic rings. Specific examples of such oxygenated heterocyclic rings are, for example, tetrahydrofuranyl, tetrahydropyranyl, 1,4-dioxanyl, and like groups.

“(C₁-C₆)alkoxy” refers to groups of the formula (C₁-C₆)alkyl-O—, where (C₁-C₆)alkyl as defined herein. Specific alkoxy groups include, by way of example, methoxy, ethoxy, propoxy, iso-propoxy, n-butoxy, tert-butoxy, sec-butoxy, n-pentoxy, n-hexoxy, 1,2-dimethylbutoxy, and like groups.

“Substituted (C₁-C₆)alkoxy” refers to a substituted (C₁-C₆)alkyl-O— group wherein substituted (C₁-C₆)alkyl is as defined above. Substituted (C₁-C₆)alkoxy is exemplified by groups such as O—CH₂CH₂—NRₐR_{b}, O—CH₂CH₂—CHRₐR_{b}, or O—CH₂—CHOH—CH₂—OH, and like groups. Specific substituted (C₁-C₆)alkoxy groups are (C₁-C₆)alkyl substituted with one or more substituents of the formula —NRₐR_{b} where Rₐ and R_{b} together with the nitrogen to which they are attached form of a heterocyclic ring. Specific examples of such heterocyclic rings include piperazino, pyrrolidino, piperidino, morpholino, or thiomorpholino. Other specific substituted (C₁-C₆)alkoxy groups are (C₁-C₆)alkoxy groups substituted with one or more carbon-linked oxygen containing heterocyclic rings. Specific examples of specific oxygenated heterocyclic ring substituents are, for example, tetrahydrofuranyl, tetrahydropyranyl, 1,4-dioxanyl, and like groups. Specific examples of such oxygenated heterocyclic rings are, for example, tetrahydrofuranyl, tetrahydropyranyl, 1,4-dioxanyl, and like groups.

“(C₁-C₆)alkanoyloxy” includes, by way of example, formyloxy, acetoxy, propanoyloxy, iso-propanoyloxy, n-butanoyloxy, tert-butanoyloxy, sec-butanoyloxy, n-pentanoyloxy, n-hexanoyloxy, 1,2-dimethylbutanoyloxy, and like groups.

“Substituted (C₁-C₆)alkanoyloxy” refers to a (C₁-C₆)alkanoyloxy group wherein one or more (e.g. 1 or 2) carbon atoms in the alkyl chain have been replaced with a heteroatom independently selected from —O—, —S— and NR— (where R is hydrogen or C₁-C₆alkyl) and/or wherein the alkyl group is substituted with from 1 to 5 substituents independently selected from cycloalkyl, substituted cycloalkyl, (C₁-C₆)alkoxycarbonyl (e.g. —CO₂Me), cyano, halo, hydroxy, oxo (═O), carboxy (COOH), aryloxy, heteroaryloxy, heterocyclooxy, nitro, and —NR^{a}R^{b}, wherein R^{a} and R^{b} may be the same or different and are chosen from hydrogen, alkyl, arylalkyl, heteroarylalkyl, heterocycloalkyl, cycloalkyl, substituted cycloalkyl, aryl, heteroaryl and heterocyclic. Substituted (C₁-C₆)alkanoyloxy is exemplified by groups such as —O—C(═O)CH₂—NRₐR_{b}, and O—C(═O)—CHOH—CH₂—OH. Specific substituted (C₁-C₆)alkanoyloxy groups are groups wherein the alkyl group is substituted with one or more nitrogen and oxygen containing heterocyclic rings such as piperazino, pyrrolidino, piperidino, morpholino, thiomorpholino, tetrahydrofuranyl, tetrahydropyranyl, 1,4-dioxanyl, and like groups.

Aryl denotes a phenyl radical or an ortho-fused bicyclic carbocyclic radical having about nine to ten ring atoms in which at least one ring is aromatic. Examples of aryl include phenyl, indenyl, and naphthyl.

Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl include furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) and quinolyl (or its N-oxide).

The term “heterocycle” refers to a monovalent saturated or partially unsaturated cyclic non-aromatic group which contains at least one heteroatom, preferably 1 to 4 heteroatoms, selected from nitrogen (NRₓ, wherein Rₓ is hydrogen, alkyl, or a direct bond at the point of attachment of the heterocycle group), sulfur, phosphorus, and oxygen within at least one cyclic ring and which may be monocyclic or multi-cyclic. Such heterocycle groups preferably contain from 3 to 10 atoms. The point of attachment of the heterocycle group may be a carbon or nitrogen atom. This term also includes heterocycle groups fused to an aryl or heteroaryl group, provided the point of attachment is on a non-aromatic heteroatom-containing ring. Representative heterocycle groups include, by way of example, pyrrolidinyl, piperidinyl, piperazinyl, imidazolidinyl, morpholinyl, indolin-3-yl, 2-imidazolinyl, 1,2,3,4-tetrahydroisoquinolin-2-yl, quinuclidinyl and the like.

“Aryloxy” refers to a group of the formula aryl-O—, where aryl is as defined herein. Examples of aryloxy groups include, phenoxy and 1-naphthyloxy. “Heteroaryloxy” refers to a group of the formula heteroaryl-O—, where heteroaryl is as defined herein. Examples of heteroaryloxy groups include, 3-piperidyloxy, 3-furyloxy, and 4-imidazoyloxy.

“Heterocyclooxy” refers to a group of the formula heterocycle-O—, where heterocycle is as defined herein. Examples of heterocyclooxy groups include, 4-morpholinooxy and 3-tetrahydrofuranyloxy.

“Arylalkyl” refers to a group of the formula aryl-(C₁-C₆)alkyl-, where aryl and (C₁-C₆)alkyl are as defined herein.

“Heteroarylalkyl” refers to a group of the formula heteroaryl-(C₁-C₆)alkyl -, where heteroaryl and (C₁-C₆)alkyl are as defined herein. “Heterocycloalkyl” refers to a group of the formula heterocycle-(C₁-C₆)alkyl -, where heterocycle and (C₁-C₆)alkyl are as defined herein.

“Effective amount” or “therapeutically effective amount” of a compound refers to a nontoxic but sufficient amount of the compound to provide the desired therapeutic or prophylactic effect to most patients or individuals. In the context of treating cancer, a nontoxic amount does not necessarily mean that a toxic agent is not used, but rather means the administration of a tolerable and sufficient amount to provide the desired therapeutic or prophylactic effect to a patient or individual. The effective amount of a pharmacologically active compound may vary depending on the route of administration, as well as the age, weight, and sex of the individual to which the drug or pharmacologically active agent is administered Those of skill in the art given the benefit of the present disclosure can easily determine appropriate effective amounts by taking into account metabolism, bioavailability, and other factors that affect plasma levels of a compound following administration within the unit dose ranges disclosed further herein for different routes of administration.

“Treatment” or “treating” refers to any manner in which the symptoms of a condition, disorder or disease are ameliorated or otherwise beneficially altered. In the context of treating the cancers disclosed herein, the cancer can be onset, relapsed or refractory. Full eradication of the condition, disorder or disease is not required. Amelioration of symptoms of a particular disorder refers to any lessening of symptoms, whether permanent or temporary, that can be attributed to or associated with administration of a therapeutic composition of the present invention or the corresponding methods and combination therapies. Treatment also encompasses pharmaceutical use of the compositions in accordance with the methods disclosed herein.

“Mammal” as used herein includes humans.

“Prodrug” as used herein refers to any compound that when administered to a biological system generates the drug substance, i.e. active ingredient of formula I or a salt thereof, as a result of spontaneous chemical reaction(s), enzyme catalyzed chemical reaction(s), photolysis, and/or metabolic chemical reaction(s). A prodrug is thus a modified analog or latent form of a therapeutically-active compound.

“Solubilizing group(s) R_{z}” is a substituent that increases the water solubility of the compound of formula I compared to the corresponding compound lacking the R substituent. Examples of solubilizing groups include substituents independently selected from substituted (C₁-C₆)alkyl, (C₁-C₆)alkoxycarbonyl (e.g. —CO₂Me), cyano, halo, hydroxy, oxo carboxy (COOH), aryloxy, heteroaryloxy, heterocyclooxy, nitro, and —NRₐR_{b}, wherein Rₐ and R_{b} may be the same or different and are chosen from hydrogen, alkyl, arylalkyl, heteroarylalkyl, heterocycloalkyl, cycloalkyl, substituted cycloalkyl, aryl, heteroaryl and heterocyclic.

Specific R₁ groups are exemplified by, for example, groups such as hydroxymethyl, hydroxyethyl, hydroxypropyl, 2-aminoethyl, 3-aminopropyl, 2-methylaminoethyl, 3-dimethylaminopropyl, 2-carboxyethyl, hydroxylated alkyl amines, such as 2-hydroxyaminoethyl, and like groups. Other specific R₁ groups are (C₁-C₆)alkyl groups substituted with one or more substituents of the formula —NRₐR_{b} where Rₐ and R_{b} together with the nitrogen to which they are attached form a nitrogen containing heterocyclic ring, or (C₁-C₆)alkyl groups substituted with one or more oxygen containing heterocyclic rings. Specific examples of such heterocyclic rings include piperazino, pyrrolidino, piperidino, morpholino, or thiomorpholino. Still other specific R₁ groups are (C₁-C₆)alkyl groups substituted with one or more carbon-linked oxygen containing heterocyclic rings. Specific examples of such oxygenated heterocyclic rings are, for example, tetrahydrofuranyl, tetrahydropyranyl, 1,4-dioxanyl, and like groups.

Specific and specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl.

Specifically, (C₁-C₆)alkoxy can be methoxy, ethoxy, propoxy, isopropoxy, butoxy, iso-butoxy, sec-butoxy, pentoxy, 3-pentoxy, or hexoxy.

A specific value for A is CH.

Another specific value for A is N.

A specific value for B is N.

Another specific value for B is CH.

A specific value for W is N.

Another specific value for W is CH.

A specific value for Y is OH.

Another specific value for Y is (C₁-C₆)alkoxy.

Another specific value for Y is —OCH₃.

Another specific value for Y is substituted (C₁-C₆)alkoxy.

Another specific value for Y is —OCH₂CH₂OH.

Another specific value for Y is —OCH₂CH₂OCH₂CH₃.

Another specific value for Y is —O—CH₂—CHOH—CH₂—OH.

Another specific value for Y is —O—CH₂CH₂—NRₐR_{b} wherein Rₐ and R_{b} are hydrogen or (C₁-C₆)alkyl.

Another specific value for Y is —O—CH₂CH₂—NRₐR_{b} wherein Rₐ and R_{b} together with the nitrogen to which they are attached form a piperazino, pyrrolidino, piperidino, morpholino, or thiomorpholino ring.

Another specific value for Y is —O—C(═O)CH₂—NRₐR_{b}.

Another specific value for Y is —O—C(═O)—CHOH—CH₂—OH.

Another specific value for Y is (C₁-C₆)alkyl substituted with one or more tetrahydrofuranyl, tetrahydropyranyl, or 1,4-dioxanyl rings.

Another specific value for Y is —O—C(═O)CH₂—NRₐR_{b}.

A specific value for Z is OH.

Another specific value for Z is (C₁-C₆)alkoxy.

Another specific value for Z is OCH₃.

Another specific value for Z is substituted (C₁-C₆)alkoxy.

Another specific value for Z is —OCH₂CH₂OH.

Another specific value for Z is —OCH₂CH₂OCH₂CH₃.

Another specific value for Z is —O—CH₂—CHOH—CH₂—OH.

Another specific value for Z is —O—CH₂CH₂—NRₐR_{b} wherein Rₐ and R_{b} are hydrogen or (C₁-C₆)alkyl.

Another specific value for Z is —O—CH₂CH₂—NRₐR_{b} wherein Rₐ and R_{b} together with the nitrogen to which they are attached form a piperazino, pyrrolidino, piperidino, morpholino, or thiomorpholino ring.

Another specific value for Z is —O—C(═O)—CHOH—CH₂—OH.

Another specific value for Z is (C₁-C₆)alkyl substituted with one or more tetrahydrofuranyl, tetrahydropyranyl, or 1,4-dioxanyl rings.

Another specific value for Z is —O—C(═O)CH₂—NRₐR_{b}.

A specific value for R₃ and R₄ is H.

Another specific value for R₃ and R₄ together is ═O.

Another specific value for R₃ and R₄ together is ═S.

Another specific value for R₃ and R₄ together is ═NH.

Another specific value for R₃ and R₄ together is ^{═N—R}₂.

Another specific value for R₃ and R₄ together is ═N—R₂ where R₂ is (C₁-C₆)alkyl.

Another specific value for R₃ and R₄ together is ═N—R₂ where R₂ is substituted (C₁-C₆)alkyl.

Another specific value for R₃ is H and R₄ is (C₁-C₆)alkyl.

Another specific value for R₃ is H and R₄ is substituted (C₁-C₆)alkyl.

Another specific value for R₃ is (C₁-C₆)alkyl and R₄ is substituted (C₁-C₆)alkyl.

Another specific value for R₃ and R₄ is substituted (C₁-C₆)alkyl

A specific value for R₁ is 2-hydroxyethyl.

Another specific value for R₁ is 2-aminoethyl.

Another specific value for R₁ is 2-(N,N′-dimethylamino)ethyl.

Another specific value for R₁ is 2-(N,N′-diethylamino)ethyl.

Another specific value for R₁ is 2-(N,N′-diethanolamino)ethyl of the formula —CH₂—CH₂—N(—CH₂—CH₂—OH)₂.

Another specific value for R₁ or R₂ is a (C₁-C₆)alkyl substituted with one or more hydroxy, mercapto, carboxy, amino, piperazinyl, pyrrolidinyl, piperidinyl, morpholinyl, thiomorpholinyl, tetrahydrofuranyl, tetrahydropyranyl, or 1,4-dioxanyl groups.

Another specific value for R₁ or R₂ is a (C₁-C₆)alkyl with from 2 to 4 carbon atoms and substituted with one to two groups selected from hydroxy, mercapto, carboxy, amino, piperazinyl, pyrrolidinyl, piperidinyl, morpholinyl, thiomorpholinyl, tetrahydrofuranyl, tetrahydropyranyl, or 1,4-dioxanyl.

Another specific value for R₁ or R₂ is —CH₂CH₂—NRₐR_{b} wherein Rₐ and R_{b} are hydrogen or (C₁-C₆)alkyl.

Another specific value for R₁ or R₂ is —CH₂CH₂—NRₐR_{b} wherein Rₐ and R_{b} together with the nitrogen to which they are attached form a piperazino, pyrrolidino, piperidino, morpholino, or thiomorpholino ring.

A specific compound of formula (I) is the compound 11,12-dihydro-2,3-dimethoxy-8,9-methylenedioxy-11-[2-(dimethylamino)ethyl]-5,6,11-triazachrysen-12-one, or a pharmaceutically acceptable salt or prodrug thereof.

A specific compound of formula I is a compound of formula II:

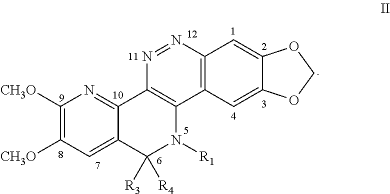

Another specific compound of formula I is a compound of formula III:

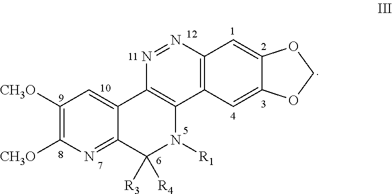

Another specific compound of formula I is a compound of formula IV:

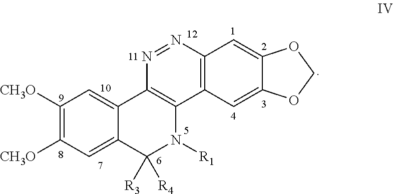

Another specific compound of formula I is a compound of formula V:

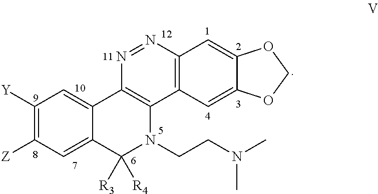

Another specific compound of formula I is a compound of formula VI:

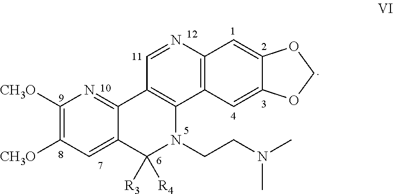

Another specific compound of formula I is a compound of formula VII:

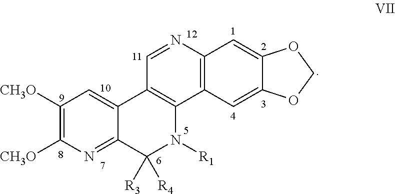

Another specific compound of formula I is a compound of formula VIII:

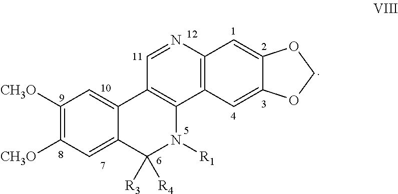

Another specific compound of formula I is a compound of formula IX:

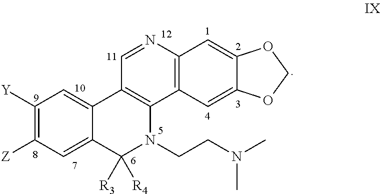

Another specific compound of formula I is any of the above compounds of formulas II-IX as a pharmaceutically acceptable salt. Specific compounds useful for the methods of treating cancer (e.g. colon cancer, non-small cell lung cancer (NSCLC), melanoma, NCI-H292 lung cancer, renal cancer, H1299 lung cancer, colorectal cancer, cervical cancer, breast cancer, and multiple myeloma) and corresponding pharmaceutical compositions of the present disclosure include 8,9-dimethoxy-2,3-methylenedioxy-5-[2-(N,N-dimethylamino)ethyl]-5H-dibenzo[c,h]1,6-naphthyridin-6-one; 8,9-dimethoxy-2,3-methylenedioxy-5-[2-(N,N-diethylamino)ethyl]-5H-dibenzo[c,h]1,6-naphthyridin-6-one; and 8,9-dimethoxy-2,3-methylenedioxy-5-[2-(N-methylamino)ethyl]-5H-dibenzo[c,h]1,6-naphthyridin-6-one; and pharmaceutically acceptable salts and prodrugs thereof A specific compound of formula I that has been found to be particularly active against colon cancer cells and multiple myeloma cells is 8,9-dimethoxy-2,3-methylenedioxy-5-[2-(N-methylamino)ethyl]-5H-dibenzo[c,h]1,6-naphthyridin-6-one (2); or a pharmaceutically acceptable salt or prodrug thereof

In one embodiment of the invention, the cancer is colon cancer, non-small cell lung cancer (NSCLC), cervical cancer, breast cancer, or multiple myeloma.

In one embodiment of the invention, the cancer is melanoma, NCI-H292 lung cancer, renal cancer, H1299 lung cancer, or colorectal cancer.

In one embodiment of the invention, the cancer is non-small cell lung cancer, melanoma, lung cancer, or renal cancer.

In one embodiment of the invention, the cancer is colorectal cancer, cervical cancer, or breast cancer.

The compounds of formula I can be prepared as described in international patent application number PCT/US02/36901, the entire content of which is hereby incorporated herein by reference.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal, for example, sodium, potassium or lithium, or alkaline earth metal, for example calcium, salts of carboxylic acids can also be made.

The compositions of the present disclosure may be formulated in a conventional manner using one or more pharmaceutically acceptable carriers or excipients. The pharmaceutically acceptable carrier can be any such carrier known in the art including those described in, for example, Remington's Pharmaceutical Sciences, Mack Publishing Co., (A. R. Gennaro edit. 1985). Pharmaceutical compositions of the compounds presently disclosed may be prepared by conventional means known in the art including, for example, mixing at least one presently disclosed compound with a pharmaceutically acceptable carrier.

The compounds presently disclosed may also be formulated for sustained delivery according to methods well known to those of ordinary skill in the art. Examples of such formulations can be found in U.S. Pat. Nos. 3,119,742, 3,492,397, 3,538,214, 4,060,598, and 4,173,626.

Thus, the active compounds of the disclosure may be formulated for oral, buccal, intranasal, parenteral (e.g., intravenous, intramuscular or subcutaneous), rectal administration, in a form suitable for administration by inhalation or insufflation, or the active compounds may be formulated for topical administration.

Thus, the present compounds may be systemically administered, for example, orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the specific methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula Ito the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of formula I in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound may conveniently be administered in unit dosage form; for example, containing 5 to 1000 mg, conveniently 10 to 750 mg, most conveniently, 50 to 500 mg of active ingredient per unit dosage form

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Test A

The ability of a compound to inhibit cancer cell growth was evaluated using the 60-cell screening assay of the DTP anticancer drug discovery program at the National Cancer Institute (United States). Results from this assay for the lukemia cell line RPMI-8266 and the colon cancer cell lines HT29 and HCT-116 are shown below.

| Cell Line | GI₅₀ | TGI | LC₅₀ |
|---|---|---|---|
| RPMI-8226 | 1.00 × 10⁻⁸ | 1.00 × 10⁻⁴ | 1.00 × 10⁻⁴ |
| HT29 | 1.30 × 10⁻⁸ | 3.21 × 10⁻⁶ | 1.46 × 10⁻⁵ |
| HCT-116 | 1.00 × 10⁻⁸ | | |

The ability of a compound to inhibit cancer cell growth can also be evaluated as described in Test B below.

### Test B

For human tumor cell CFU assays, the cell lines which grow as monolayers, MDA-MB-231, HCT116, HT29, NCI-H460, KB3-1 and KBV-1 were grown in RPMI medium (Invitrogen/Gibco, Grand Island, N.Y.) supplemented with 5% fetal bovine serum (Invitrogen/Gibco, Grand Island, NY). The RPMI-8226 cell line grows in suspension.

For human tumor cell CFU assays, RPMI-8226 cells were grown in 0.35% agar in DMEM-F12 medium supplemented with 10% fetal bovine serum over a base layer of 0.5% agar in DMEM-F12 medium supplemented with 10% fetal bovine serum

For experiments, human tumor cells (1 103) were plated in 6-well plates in medium supplemented with 5% or 10% fetal bovine serum. The compounds were tested in concentrations over the range from 0.01 to 100 nanomolar in half-log intervals covering 5 logs along with untreated control wells. In later experiments some cases the concentration ranges were refined to focus on the region of interest in the response curves. Each compound concentration was tested in duplicate wells. Cultures were exposed to the compounds continuously for 7-9 days at 37° C. in a humidified atmosphere of 5% carbon dioxide balance air. Each experiment was performed three independent times. Colonies were defined as clusters containing 30 or more cells.

For the monolayer cultures, colonies were visualized by staining with a preformulated crystal violet solution (Fisher Cat #291-472) which contained 0.41% crystal violet, 12% ethanol balance deionized water. To visualize the colonies, the medium was removed by aspiration; the monolayer was rinsed once with phosphate buffered saline which was removed by aspiration. Three drops of crystal violet solution was added to each well and the 6-well plate was rotated so that the crystal violet solution covered the surface area of each well. After 5 minutes exposure time, the wells were rinsed twice with phosphate buffered saline and the colonies were visible.

The IC₅₀ and IC90 values and the 95% confidence interval for each compound for each human tumor cell line were determined by non-linear regression analysis using SAS version 8.2 by Xian-Jie Yu, Senior Biostatistician (Stability & Statistics Department, Genzyme Corporation, Framingham, Mass.). The values were expressed as the mean values with lower and upper 95% confidence intervals in nanomolar concentrations.

The following compounds 1-4 as well as 7-ethyl-10-hydroxyl-camptothecin (SN-38, a potent topoisomerase), and topotecan were evaluated in this assay.

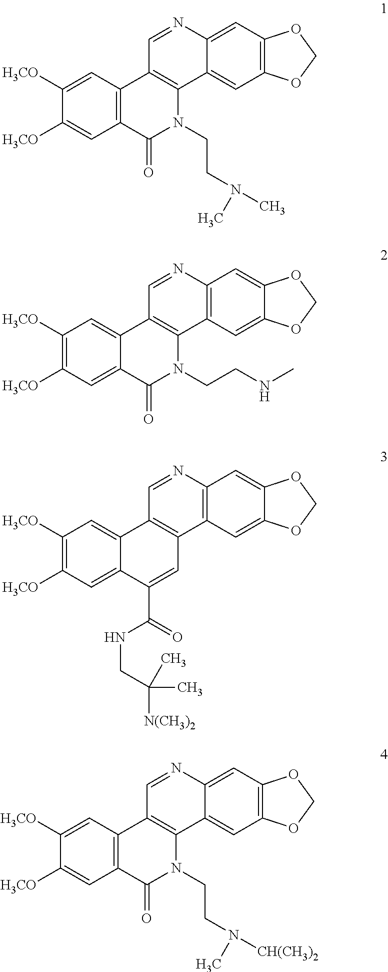

As shown in the following tables, compounds 1, 2, 3, and 4 were potent cytotoxic agents toward human tumor cells. Exposure to the compounds produced exponential killing of cells in a manner consistent with potent inhibition of a critical molecular target. With all six compounds tested, concentrations killing 50% and 90% of the cells were readily achieved. The human tumor cell IC₅₀ and IC90 values and lower and upper 95% confidence intervals for the six compounds are presented in nanomolar concentrations below.

IC₅₀ Values nM (95% Lower and Upper Confidence Intervals)

| | MDA-MB-231 | HCT116 Human | HT29 Human | RPMI-8226 | |
|---|---|---|---|---|---|
| | Human Breast | Colon | Colon | Human Multiple | |
| Compound | Carcinoma | Carcinoma | Carcinoma | Myeloma | |
| 2 | 0.2 (0.1-0.3) | 0.9 (0.5-1.4) | 0.15 (0.1-0.3) | 0.2 | (0.13-0.3) |
| 1 | 0.3 (0.2-0.6) | 1.7 (1.4-2.2) | 1.3 (1.1-1.6) | 1.8 | (1.4-2.2) |
| 3 | 0.5 (0.3-0.9) | 0.4 (0.3-0.6) | 0.5 (0.4-0.6) | 0.7 | (0.6-0.8) |
| 4 | 0.3 (0.2-0.5) | 1.2 (1.1-1.3) | 0.5 (0.4-0.7) | 0.4 | (0.3-0.5) |
| SN-38 | 0.7 (0.5-0.9) | 2.7 (2.4-3.2) | 0.5 (0.4-0.7) | 0.9 | (0.7-1.1) |
| Topotecan | 5.6 (4.6-7.2) | 8.5 (6.7-1.1) | 2.9 (2.2-3.9) | 12.7 | (10.7-15.5) |

### IC₅₀ Values nM (95% Lower and Upper Confidence Intervals)

| | NCI-H460 Human | KB3-1 HeLa | | KBH5.0 | KB-V1 | |
|---|---|---|---|---|---|---|
| | Non-small Cell Lung | Human Cervical | | BCRP+ KB3-1 | MDR1+ KB3-1 | |
| Compound | Carcinoma | Carcinoma | | Subline | Subline | |
| 2 | 1.2 (0.9-2.2) | 1.7 | (1.3-2.5) | 1.0 (0.6-1.7) | 2.0 | (1.3-3.1) |
| 1 | 2.3 (1.3-4.0) | 1.5 | (1.1-2.3) | 1.8 (1.2-2.8) | 1.8 | (1.2-2.9) |
| 3 | 0.9 (0.7-1.2) | 0.8 | (0.6-1.1) | 0.6 (0.4-1.1) | 0.6 | (0.4-1.1) |
| 4 | 3.4 (2.0-5.0) | 1.0 | (0.6-1.7) | 1.3 (1.0-1.8) | 1.4 | (1.1-2.0) |
| SN-38 | 4.7 (3.5-6.5) | 5.3 | (2.8-11.4) | 6.1 (4.4-8.8) | 15 | (11.1-21.4) |
| Topotecan | 18.2 (9.5-36.3) | 32.7 | (18.8-61.6) | 32.0 (23.7-44.2) | 75 | (45.7-133.4) |

### IC₉₀ Values nM (95% Lower and Upper Confidence Intervals)

| | MDA-MB-231 | HCT116 Human | HT29 Human | RPMI-8226 | |
|---|---|---|---|---|---|
| | Human Breast | Colon | Colon | Human Multiple | |
| Compound | Carcinoma | Carcinoma | Carcinoma | Myeloma | |
| 2 | 0.7 (0.5-0.9) | 2.8 (1.3-4.7) | 0.9 (0.5-1.2) | 0.8 | (0.63-1.0) |
| 1 | 1.2 (0.7-1.7) | 5.6 (4.2-7.0) | 4.3 (3.4-5.1) | 5.8 | (4.5-7.0) |
| 3 | 0.9 (0.5-1.4) | 1.7 (1.1-2.2) | 1.5 (1.2-2.0) | 2.5 | (2.2-3.0) |
| 4 | 1.0 (0.6-1.3) | 4.4 (4.0-5.1) | 1.9 (1.3-2.2) | 1.5 | (1.0-1.9) |
| SN-38 | 2.0 (1.5-2.5) | 8.4 (7.1-9.8) | 1.8 (1.2-2.3) | 3.0 | (2.4-3.6) |
| Topotecan | 19.5 (15.0-24.0) | 26.3 (19.3-33.1) | 11.2 (8.0-14.1) | 43.2 | (34.7-51.3) |

### IC₉₀ Values nM (95% Lower and Upper Confidence Intervals)

| | NCI-H460 Human | KB3-1 HeLa | KBH5.0 | | KB-V1 | |
|---|---|---|---|---|---|---|
| | Non-small Cell Lung | Human Cervical | BCRP+ KB3-1 | | MDR1+ KB3-1 | |
| Compound | Carcinoma | Carcinoma | Subline | | Subline | |
| 2 | 5.0 (2.5-7.0) | 7.7 (6.0-9.1) | 3.0 | (2.0-6.0) | 8.3 | (6.0-10.1) |
| 1 | 6.2 (2.9-9.1) | 5.2 (3.2-7.2) | 5.8 | (3.5-8.0) | 5.7 | (3.2-7.2) |
| 3 | 3.0 (2.1-4.0) | 2.8 (2.2-4.3) | 2.3 | (1.2-3.2) | 2.8 | (2.0-5.0) |
| 4 | 11.0 (6.3-15.0) | 4.0 (2.1-6.8) | 6.0 | (3.5-7.4) | 6.9 | (4.6-8.1) |
| SN-38 | 13.3 (9.0-17.4) | 19.1 (8.0-30.5) | 18.8 | (12.2-25.1) | 55 | (37.2-72.4) |
| Topotecan | 52.5 (21.4-83.2) | 107.2 (50.7-162.2) | 114.8 | (78.5-147.9) | 257 | (128.8-384.6) |

The activity of representative compounds was evaluated in tumor xenograph models as described below.

### Compound 2 Citrate Salt vs. HCT-116 Human Colon Tumor Xenograft Model

Study Objective: The objective of this study was to determine the efficacy of Compound 2 citrate salt and an experimental compound against the HCT-116 human colon tumor xenograft model. Irinotecan served as the positive control.

Materials and Methods:

Test and Control Article Formulation Preparation: On each day of dosing, the test article,

Compound 2 citrate salt, was weighed out and dissolved in the appropriate volume of D5W. The positive control article (irinotecan) dosing solution was prepared on each day of dosing by diluting an irinotecan stock solution with an appropriate volume of D5W. A 10 mL/kg dose volume was administered to all animals.

Xenografts: Male nude (nu/nu) mice were implanted subcutaneously in the axilla region by trocar with fragments of HCT-1 16 human colon tumors harvested from subcutaneously growing tumors in nude mice hosts. The mice were approximately 4 weeks of age and weighed 18-20 g at the time of tumor implantation. When the tumors were 220-23 5 mm³ in size (11 days following implantation), the animals were pair-matched into treatment and control groups.

Dose Administration and Schedule: Beginning on Day 11, groups of 8 male nude (nu/nu) mice were administered Compound 2citrate salt IV at doses of 0 (untreated control), 0 (vehicle control), 1.36, 2.72, or 5.44 mg/kg/day (4.1, 8.2, or 16.3 mg/m²) on a qod×3 weekly for 2 cycles dosing schedule. Another group of 8 male nude (nu/nu) mice were administered irinotecan, the positive control, IV at a dose of 60 mg/kg/day on a q4d×3 dosing schedule.

Body Weight: All mice were individually weighed prior to each dose (for dose calculation purposes only) and twice weekly.

Tumor Measurements and Study Endpoints: Tumor volumes were measured twice weekly. Mice were evaluated for two tumor growth endpoints, percent TGI (T/C %) and TGD (T-C days) with corresponding ILS.

Results: Compound 2citrate salt at 1.36 and 2.72 mg/kg/day resulted in low and moderate TGI activity (T/C=45.0% and 3 3.2%, respectively). At the second evaluation point, Compound 2 citrate salt at the low dose resulted in low TGD activity (T-C=18 days corresponding to a 1.6-fold ILS. The medium dose exhibited high TGD activity (TGD=>34 days) corresponding to a >2.2-fold ILS. At the conclusion of the study, Day 62, 50% of the mice were survivors. The high dose of Compound 2 citrate salt (5.44 mg/kg/day), resulted in >30% weight loss and ⅝ toxic deaths.

Irinotecan exhibited moderate TGI activity (T/C %=3 9.2%) and borderline low TGD activity (T-C=14 days) corresponding to a 1.5-fold ILS. This agent was tolerated well at the dose level tested.

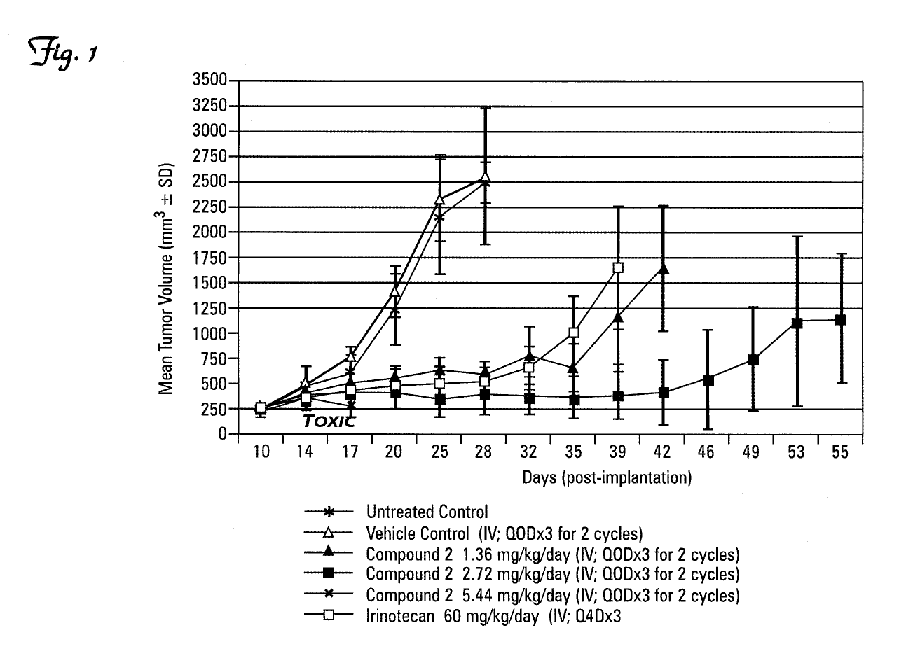

As evidenced by the TGIs and delays in tumor growth, Compound 2 citrate salt exhibited activity against the HCT-1 16 human colon tumor xenograft model. Compound 2 citrate salt was superior to the control irinotecan (See FIG. 1).

Compound 2 Citrate Salt vs. NCI-460 Human Non-Small Cell Lung Carcinoma Xenograft Model

Study Objective: The objective of this study was to determine the efficacy of Compound 2 citrate salt against the NCI-H460 human non-small cell lung carcinoma xenograft model. Docetaxel served as the positive control.

Test and Control Article Formulation Preparation: On each day of dosing, the test article, Compound 2 citrate salt, was weighed out and dissolved in the appropriate volume of D5W. The positive control article, docetaxel was weighed out and dissolved in the appropriate volume of ethanol, and once in solution, the appropriate volume of CremophorEL and saline were added to yield a solution. A 10 mL/kg dose volume was administered to all animals.

Materials and Methods:

Xenografts: Male nude (nu/nu) mice were implanted subcutaneously in the axilla region by trocar with fragments of NCI-H460 human non-small cell tumors harvested from subcutaneously growing tumors in nude mice hosts. The mice were approximately 4 weeks of age and weighed 20-25 g at the time of tumor implantation. When the tumors were 195-22 1 mm³ in size (10 days following implantation), the animals were pair-matched into treatment and control groups.

Dose Administration and Schedule: Beginning on Day 10, groups of 8 male nude (nu/nu) mice were administered Compound 2 citrate salt IP at doses of 0 (untreated control), 0 (vehicle control), 0.68, 1.36, or 2.72 mg/kg (2.0, 4.1, or 1.36 mg/m²) on a qod×3 weekly for 2 cycles dosing schedule. Another group of 8 male nude (nu/nu) mice were administered docetaxel IV at a dose of 20 mg/kg/day on a qod×3 dosing schedule.

Body Weight: All mice were individually weighed prior to each dose (for dose calculation purposes only) and twice weekly.

Tumor Measurements and Study Endpoints: Tumor volumes were measured twice weekly.

Mice were evaluated for two tumor growth endpoints, percent TGI (T/C %) and TGD (T-C days) with corresponding ILS.

Results and Conclusions: Compound 2 citrate salt exhibited activity against the NCI-H460 human non-small cell lung carcinoma xenograft model at the 2.72 mg/kg/day dose only. Compound 2 citrate salt at 2.72 mg/kg/day exhibited moderate TGI activity (T/C=35.1%) and high TGD activity (T-C=24 days) which corresponded to a 2.0-fold ILS. All of the dosages were well tolerated with <20% body weight loss and no toxic deaths.

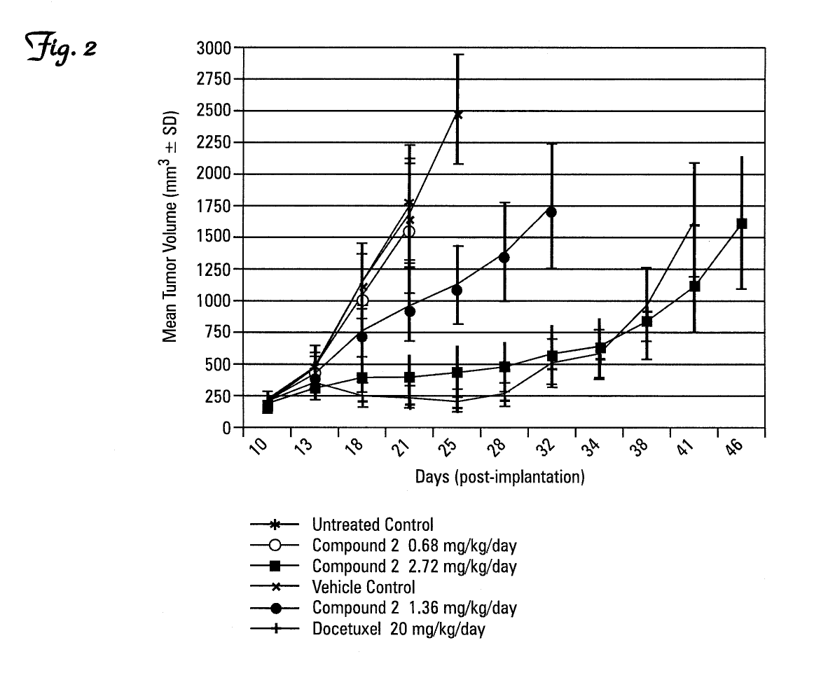

Docetaxel served as the positive control and exhibited moderate TGI activity (T/C=22.7%) and moderate TGD activity (T-C=21 days). At 20 mg/kg/day, this agent produced excessive weight loss (>20%), reaching a maximum weight loss of 26.4% on Day 25. Despite the extreme weight loss, there were no toxic deaths and the animals recovered the weight loss within 13 days. The test compound proved to be effective against the NCI-H460 human non-small cell lung carcinoma xenograft model. When compared to docetaxel, Compound 2 citrate salt proved to be slightly superior (see FIG. 2).

Comparison Dose Schedule Study of Compound 2 Citrate Salt in the NCI-H460 Human Non-Small Cell Lung Carcinoma Xenograft Model

Study Objective: The purpose of this study was to determine the efficacy of Compound 2 citrate salt administered on three dosing schedules against the NCI-H460 human non-small cell lung carcinoma xenograft model. Irinotecan served as the positive control.

Test and Control Article Formulation Preparation: On each day of dosing, the test article, Compound 2 citrate salt, was weighed out and dissolved in the appropriate volume of D5W. The positive control article, irinotecan was reconstituted from a stocksolution to the appropriate concentration with D5W. A 10 mL/kg dose volume was administered to all animals.

Materials and Methods:

Xenografts: Male nude (nu/nu) mice were implanted subcutaneously in the axilla region by trocar with fragments of NCI-H460 human non-small cell tumors harvested from subcutaneously growing tumors in nude mice hosts. The mice were approximately 5 weeks of age and weighed 22-25 g at the time of tumor implantation. When the tumors were 207-2 19 mm³ in size (10 days following implantation), the animals were pair-matched into treatment and control groups.

Dose Administration and Schedule: Beginning on Day 10, groups of 9 male nude (nu/nu) mice were administered Compound 2 citrate salt IP at doses of 0 (untreated control), 0 (vehicle control), and 2.72 mg/kg/day (8.2 mg/m²/day) on a qod×3 weekly for 2 cycles dosing schedule; 3.27 mg/kg/day (9.8 mg/m²/day) on a qd×5 dosing schedule; or 4.90 mg/kg/day (14.7 mg/m²/day) on an q4d×5 dosing schedule. Another group of 9 male nude (nu/nu) mice were administered irinotecan IP at a dose of 60 mg/kg/day on a q4d×3 and on a qod×3 weekly for 2 cycles dosing schedule.

Body Weight: All mice were individually weighed prior to each dose (for dose calculation purposes only) and twice weekly.

Tumor Measurements and Study Endpoints: Tumor volumes were measured twice weekly. Mice were evaluated for two tumor growth endpoints, percent TGI (T/C %) and TGD (T-C days) with corresponding ILS.

Results and Conclusions: Compound 2 citrate salt exhibited activity against the NCI-H460 human non-small cell lung carcinoma xenograft model. Compound 2 administered on the qod×3 weekly for 2 cycles and qd×5 dosing regimens exhibited moderate TGI activity (T/C=17.4-25.8%) and high TGD activity (T-C=29-42 days) corresponding to a 2.5-3.1-fold ILS. All of the dosages were tolerated, except for Compound 2 citrate salt administered at 4.90 mg/kg/day on a q4d×5 schedule. This group experienced a maximum weight loss of 24.2% on Day 34, which was not completely recovered at the time of study termination.

Irinotecan served as the positive control and was tested on the laboratory's standard schedule of q4d×3, and a schedule to mimic that of the test compounds, qod×3 weekly for 2 cycles. Irinotecan administered on the q4d×3 schedule exhibited moderate TGI activity (T/C=3 5.8%) and moderate TGD activity (T-C=14 days) corresponding to a 1.7-fold ILS. On the qod×3 weekly for 2 cycles schedule, irinotecan exhibited moderate TGI activity (T/C=19.0%) and high TGD activity (T-C=29 days) corresponding to a 2.5-fold ILS.

Both schedules were well tolerated.

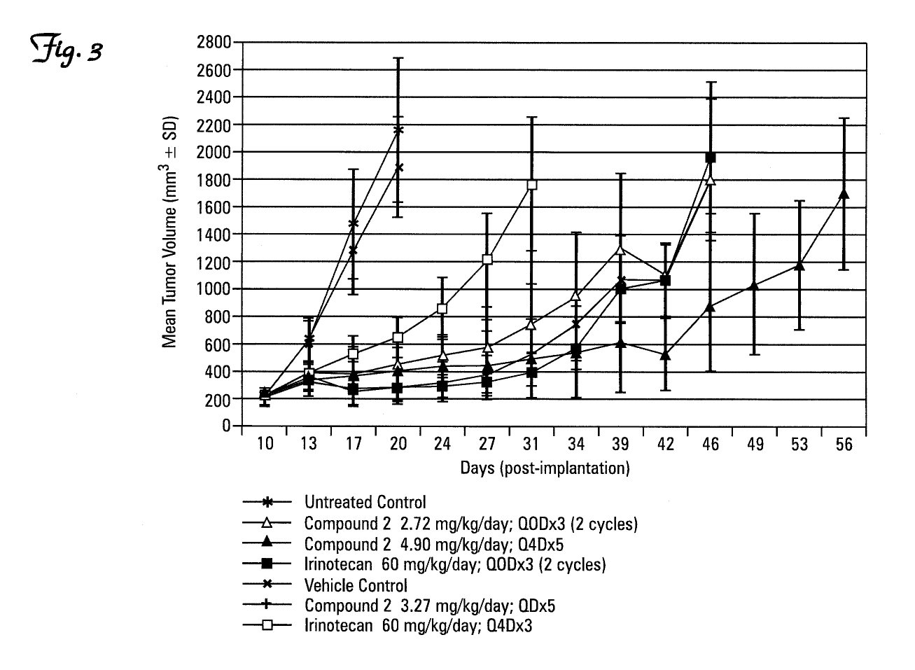

As evidenced by the TGIs and delays in tumor growth, all of the treatment groups had good antitumor activity in the NCI-H460 human non-small cell lung carcinoma xenograft model. When compared to irinotecan, Compound 2 citrate salt had comparable activity to slightly superior activity (see FIG. 3).

### Compound 2 Citrate Salt vs. HT-29 Human Colon Tumor Model

Study Objective: The objective of this study was to determine the efficacy of Compound 2 citrate salt and other experimental compounds against the HT-29 human colon tumor xenograft model. Irinotecan served as the positive control.

Test and Control Article Formulation Preparation: On each day of dosing, the test article, Compound 2 citrate salt, was weighed out and dissolved in the appropriate volume of D5W. The positive control article, irinotecan was reconstituted from a stock solution to the appropriate concentration with D5W. A 10 mL/kg dose volume was administered to all animals.

Xenografts: Male nude (nu/nu) mice were implanted subcutaneously in the axilla region by trocar with fragments of HT-29 human colon tumors harvested from subcutaneously growing tumors in nude mice hosts. The mice were approximately 5 weeks of age and weighed 20-22 g at the time of tumor implantation. When the tumors were 205-230 mm³ in size (18 days following implantation) the animals were pair-matched into treatment and control groups.

Dose Administration and Schedule: Beginning on Day 18, groups of 9 male nude (nu/nu) mice were administered Compound 2 citrate salt IP at doses of 0 (untreated control) and 0 (vehicle control), 1.36, 2.72, or 4.08 mg/kg/day (4.1, 8.2, 12.2 mg/m²/day) on a qod×3 weekly for 2 cycles dosing schedule. Another group of 9 male nude (nu/nu) mice were administered irinotecan IV at a dose of 60 mg/kg/day on a q4d×3.

Body Weight: All mice were individually weighed prior to each dose (for dose calculation purposes only) and twice weekly.

Tumor Measurements and Study Endpoints: Tumor volumes were measured twice weekly. Mice were evaluated for two tumor growth endpoints, percent TGI (T/C %) and TGD (T-C days) with corresponding ILS.

Results and Conclusions: Compound 2 citrate salt exhibited activity at doses of 2.72 and 4.08 mg/kg/day. Compound 2 citrate salt administered at 2.72 mg/kg/day resulted in low TGI activity (T/C=50.1%) and a TGD of 16 days when compared to the untreated control group. Although this dose resulted in a delay in tumor growth, the difference from the control group was not substantial enough to be considered active. The high dose of 4.08 mg/kg/day resulted in moderate TGI activity (T/C=18.9%) and borderline moderate TGD activity (T-C=31 days) corresponding to a 1.7-fold ILS. Compound 2 citrate salt was well tolerated at the dose levels tested.

Irinotecan exhibited low TGI (T/C=52.7%) and no TGD was observed. Irinotecan was well tolerated at the dose level tested.

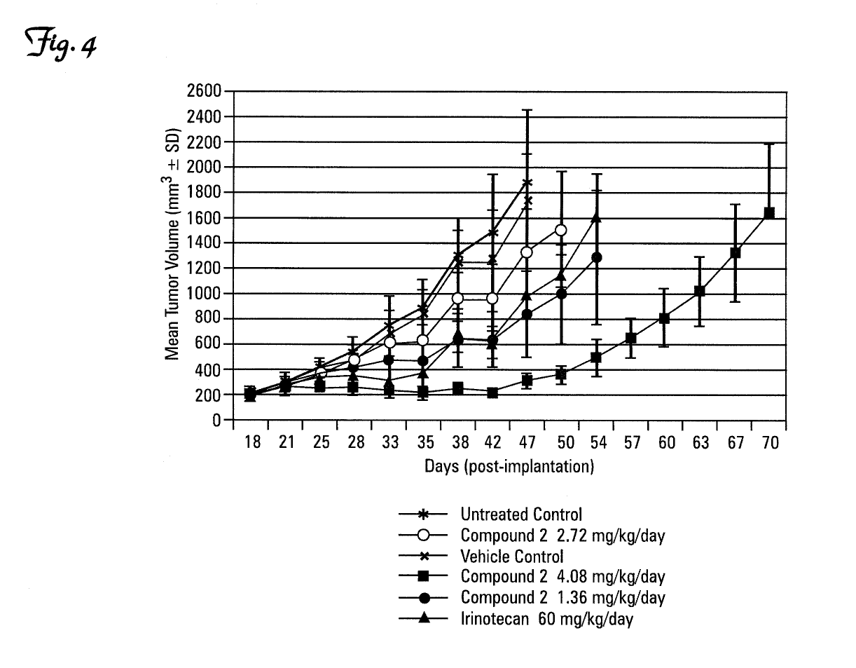

Compound 2 citrate salt was effective against the HT-29 human colon xenograft line. When compared to irinotecan, Compound 2 citrate salt was slightly superior in activity (see FIG. 4).

### Compound 2 Citrate Salt vs. NCI-H460 Human Non-Small Cell Lung Carcinoma Xenograft Model

Study Objective: The objective of this study was to determine the efficacy of Compound 2 citrate salt against the NCI-H460 human non-small cell lung carcinoma xenograft model. Pemetrexed, topotecan, and cisplatin served as the positive controls.

Test and Control Article Formulation Preparation: On each day of dosing, the test article, Compound 2 citrate salt, was weighed out and dissolved in the appropriate volume of D5W. On Day 1 of dosing, the pemetrexed stock was reconstituted with saline to yield the appropriate concentration of dosing solution. On each day of dosing, a vial of topotecan was reconstituted with sterile water for injection and then diluted to appropriate concentration with saline. On each day of dosing cisplatin was weighed out and dissolved in the appropriate volume of saline. A 10 mL/kg dose volume was administered to all animals.

Materials and Methods:

Xenografts: Male nude (nu/nu) mice were implanted subcutaneously in the axilla region by trocar with fragments of NCI-H460 human non-small cell tumors harvested from subcutaneously growing tumors in nude mice hosts. The mice were approximately 5-6 weeks of age and weighed 22-25 g at the time of tumor implantation. When the tumors were 248-270 mm³ in size (11 days following implantation), the animals were pair-matched into treatment and control groups.

Dose Administration and Schedule: Beginning on Day 11, groups of 8 male nude (nu/nu) mice were administered Compound 2 citrate salt IP at doses of 0 (untreated control) and 0 (vehicle control), 2.04 and 2.72 mg/kg/day (6.1 and 8.2 mg/m²day) on a qod×3 weekly for 2 cycles dosing schedule and at doses of 2.59 and 3.27 mg/kg/day (7.8 and 9.8 mg/m²/day) on a qd×5 dosing schedule. Additional groups of 8 male nude mice were administered pemetrexed IP at doses of 100 and 150 mg/kg/day , topotecan IP at doses of 2 and 2.5 mg/kg/day, and cisplatin IP at doses of 0.75 and 1.5 mg/kg/day on a qd×5 dosing schedule.

Body Weight: All mice were individually weighed prior to each dose (for dose calculations purposes only) and twice weekly.

Tumor Measurements and Study Endpoints: Tumor volumes were measured twice weekly. Mice were evaluated for two tumor growth endpoints, percent TGI (T/C %) and TGD (T-C days) with corresponding ILS.

Results and Conclusions: On the qod×3 weekly for 2 cycles dosing regimen, Compound 2 citrate salt was active at 2.04 and 2.72 mg/kg/day exhibiting low-to- moderate TGI activity (T/C=40.0-55.2%) and high TGD activity (T-C=24-31 days) corresponding to a >2.0-fold ILS. At 2.72 mg/kg/day, this agent produced excessive weight loss (>20%), reaching a maximum weight loss of 22.3% on Day 22. Despite the extreme weight loss, there were no toxic deaths. At the time of study termination, Day 53, the animals had recovered approximately 12% of the weight loss. On Day 53, 3 of 8 animals had not yet reached the study endpoint of 2000 mm³. The mean tumor volume of these 3 animals was 1583 mm³.

On the qd×5 dosing schedule, Compound 2 citrate salt was active at the dosages tested exhibiting moderate TGI (T/C=30.5% and 33.5%) at 2.59 and 3.27 mg/kg/day, respectively. At the second evaluation point, both dosages were highly active with a TGD of 28 days corresponding to a >2.0-fold ILS. The dosages were well tolerated (<20% body weight loss) and resulted in no toxic deaths. At 3.27 mg/kg/day, there were 3 of 8 animals that had not yet reached the study endpoint of 2000 mm³. The mean tumor volume of these 3 animals was 1722 mm³

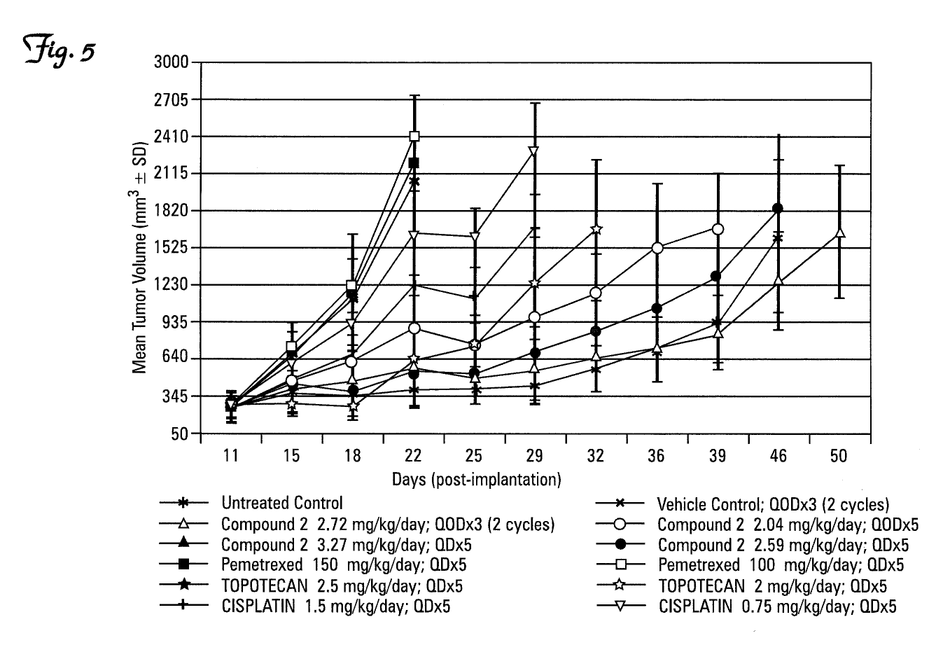

Pemetrexed was not considered active in this study. All of the dosages were well tolerated with ≦20% body weight loss. Topotecan was not tolerated in this study, exhibiting body weight loss >30%. Cisplatin was only active at the high dose. This dose resulted in low activity at both evaluation points; all of the dosages were well tolerated. Compound 2 citrate salt proved to be effective against the NCI-H460 human non-small cell lung carcinoma xenograft model. When compared to the standard therapies, Compound 2 citrate salt compound was superior. In evaluating the different schedules among the agents, there was comparable activity (see FIG. 5).

### Compound 2 Citrate Salt vs. Comparator Agents in the MDA-MB-231 Human Breast Tumor Xenograft Model

Study Objective: The purpose of this study was to determine the efficacy of Compound 2 citrate salt and an experimental compound administered on two schedules, against the MDA-MB-23 1 human breast tumor xenograft model. Irinotecan, nabpaclitaxel, oxaliplatin, and doxorubicin served as the positive controls.

Test and Control Article Formulation Preparation: On each day of dosing, the test article, Compound 2 citrate salt, was weighed out and dissolved in the appropriate volume of D5W. The irinotecan dosing solution was prepared by adding the appropriate volume of irinotecan stock solution to the appropriate volume of D5W. The nabpaclitaxel dosing solution was prepared by adding an appropriate amount of saline. The oxaliplatin dosing stock solution was prepared by adding the appropriate volume of oxaliplatin stock to the appropriate volume of D5W. The doxorubicin dosing solution was prepared by adding the appropriate volume of doxorubicin stock to the appropriate volume of saline. A 10 mL/kg dose volume was administered to all animals.

Materials and Methods:

Xenografts: Female nude (nu/nu) mice were implanted subcutaneously in the axilla region by trocar with fragments of MDA-MB-23 1 human breast tumors harvested from subcutaneously growing tumors in nude mice hosts. The mice were approximately 5-6 weeks of age and weighed 22-25 g at the time of tumor implantation. When the tumors were 223-263 mm³ in size (18 days following implantation), the animals were pair-matched into treatment and control groups.

Dose Administration and Schedule: Beginning on Day 18 groups of 8 female nude (nu/nu) mice were administered Compound 2 citrate salt IP at doses of 0 (untreated control), 0 (saline vehicle control), 0 (D5W vehicle control), 2.04, and 2.72 mg/kg/day (61.2 and 8.16 mg/m²/day) on a qod×3 weekly for 2 cycles dosing schedule, and 3.27 mg/kg/day on a qd×5 dosing. Additional groups of 8 male nude mice were administered irinotecan IP at a dose of 60 mg/kg/day on a qod×3 weekly for 2 cycles dosing schedule, nab-paclitaxel IV at doses of 200 and 300 mg/kg/day, oxaliplatin IP at doses of 5 and 6.5 mg/kg/day, or doxorubicin IP at doses of 2.5 and 3 mg/kg/day on a qd×5 dosing schedule.

Body Weight: All mice were individually weighed prior to each dose (for dose calculations purposes only) and twice weekly.

Tumor Measurements and Study Endpoints: Tumor volumes were measured twice weekly. Mice were evaluated for two tumor growth endpoints, percent TGI (T/C %) and TGD (T-C days) with corresponding ILS.

Results and Conclusions: On the qod×3 weekly for 2 cycles dosing regimen, Compound 2 citrate salt was active at 2.04 and 2.72 mg/kg/day exhibiting moderate TGI activity (T/C=12.5%-20.9%). At the second evaluation point, this compound was highly active at 2.04 and 2.72 mg/kg/day with a TGD of 52 and >58 days, respectively which corresponded to a >2.0-fold ILS. The dosages were well tolerated exhibiting a maximum loss in body weight <7%. At the time of study termination, Day 90, 2 of 8 and 4 of 8 animals had not yet reached the study endpoint of 1500 mm³ in the 2.04 and 2.72 dose groups, respectively.

On the qd×5 dosing schedule, Compound 2 citrate salt at 3.27 mg/kg/day produced high TGI activity (T/C=9.5%) and high TGD activity (T-C=42 days) corresponding to a >2.0-fold ILS. This dose was tolerated, producing a maximum weight loss of 15.7%. There was one mouse remaining on Day 90.

In this study, irinotecan, exhibited high TGI activity (T/C=10%) and high TGD activity (T-C=38 days) corresponding to a >2.0-fold ILS. All of the dosages were well tolerated with 20% body weight loss.

At both dosages, nab-paclitaxel exhibited moderate TGI activity (T-C=14.6-19.0%) and high TGD activity (T-C=45 days) with a corresponding ILS of 2.4 days. The 200 and 300 mg/kg/day groups resulted in 1 of 8 and 2 of 8 survivors, respectively, on Day 90. Dosages were well tolerated.

Oxaliplatin was only active at the first evaluation point. Both dosages produced low TGI activity with (T/C=45.1-47.6%). There was a delay in tumor growth of 13 days, but this was not substantial enough to be considered active. All of the dosages were well tolerated.

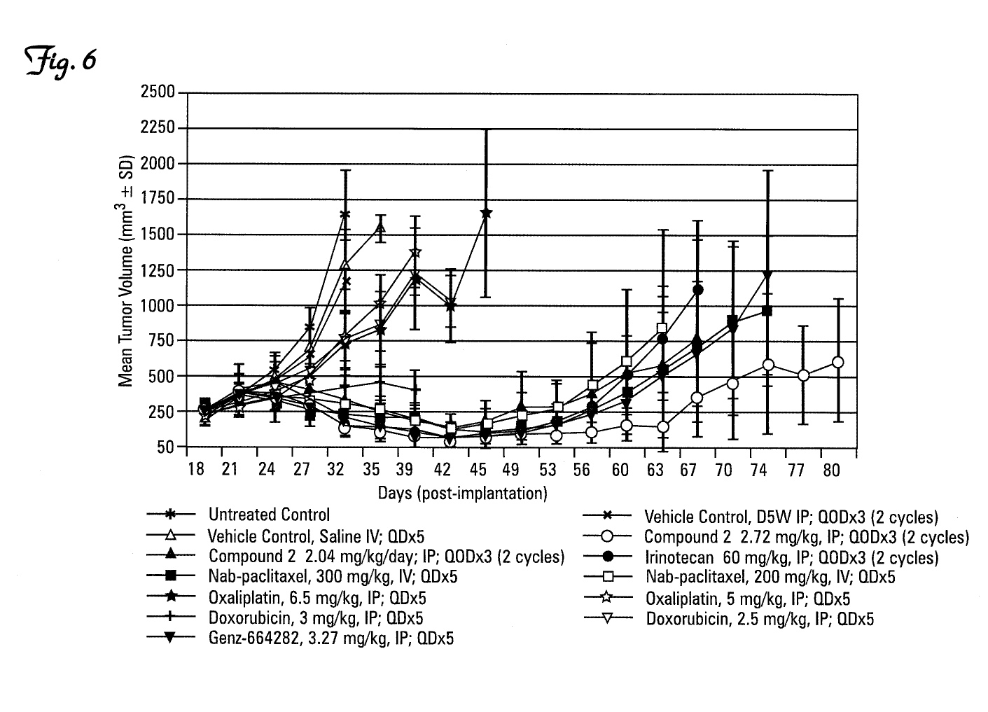

Doxorubicin was not tolerated in this study. At both dosages there were toxic deaths. Compound 2 citrate salt proved to be effective against the MDA-MB-23 1 human breast tumor xenograft model. When compared to the standard therapies, the Compound 2 citrate salt was superior to all of the standard agents, except for irinotecan which had comparable activity. The anti-tumor activity of Compound 2 citrate salt on the two different dosing schedules was comparable (see FIG. 6).

### Compound 2 After Oral Administration vs. the HCT-116 Human Colon Tumor Xenograft Model

Study Objective: The purpose of this study was to determine the oral efficacy of Compound 2 against the HCT-116 human colon tumor xenograft model. kinotecan served as the positive control.

Test and Control Article Formulation Preparation: Once a week, the test article, Compound 2 citrate salt, was weighed out and suspended in the appropriate volume of 0.5% methocellulose. On each day of dosing, the irinotecan dosing solution was prepared by adding the appropriate volume of an irinotecan stock solution to the appropriate volume of D5W. A 10 mL/kg dose volume was administered to all animals.

Materials and Methods:

Xenografts: Male nude (nu/nu) mice were implanted subcutaneously in the axilla region by trocar with fragments of HCT-1 16 human non-small cell tumors harvested from subcutaneously growing tumors in nude mice hosts. The mice were approximately 7 weeks of age and weighed 22-25 g at the time of tumor implantation. When the tumors were 177-2 16 mm³ in size (14 days following implantation), the animals were pair-matched into treatment and control groups.

Dose Administration and Schedule: Beginning on Day 14, groups of 9 male nude (nu/nu) mice were administered Compound 2 citrate salt PO at doses of 0 (untreated control), 0 (saline vehicle control), 0 (vehicle control), 0.68, 1.36, or 2.72 mg/kg/day (2.0, 4.1 or 8.2 mg/m²/day) on a qod×3 weekly for 2 cycles dosing schedule, and IV at 2.72 mg/kg/day on a qod×3 weekly for 2 cycle dosing schedule (IV group not evaluated due to dosing error). An additional group of 8 male nude mice was administered irinotecan IP at a dose of 60 mg/kg/day on a q4d 3 dosing schedule.

Body Weight: All mice were individually weighed prior to each dose (for dose calculations purposes only) and twice weekly.

Tumor Measurements and Study Endpoints: Tumor volumes were measured twice weekly. Mice were evaluated for two tumor growth endpoints, percent TGI (T/C %) and TGD (T-C days) with corresponding ILS.

Results and Conclusions: Compound 2 administered PO exhibited low-to-moderate activity at 1.36 and 2.72 mg/kg/day. The administration of 1.36 mg/kg/day showed low TGI activity (T/C=57.6%), but no effect on TGD. At 2.72 mg/kg/day, there was moderate activity in terms of TGI (T/C=3 2.2%) and low TGD activity (T-C=18 days) corresponding to a 1.5-fold ILS. The dosages were tolerated as there was <20% weight loss exhibited and no toxic deaths.

Irinotecan exhibited moderate TGI activity (T/C=3 3.8%) and moderate TGD (T-C=>18 days) corresponding to >1.5-fold ILS. At the time of study termination, Day 53, 8 of 9 animals remained (mean tumor volume=1153 mm³) and an exact TGD could not be determined. This dosage was well tolerated producing <10% body weight loss.

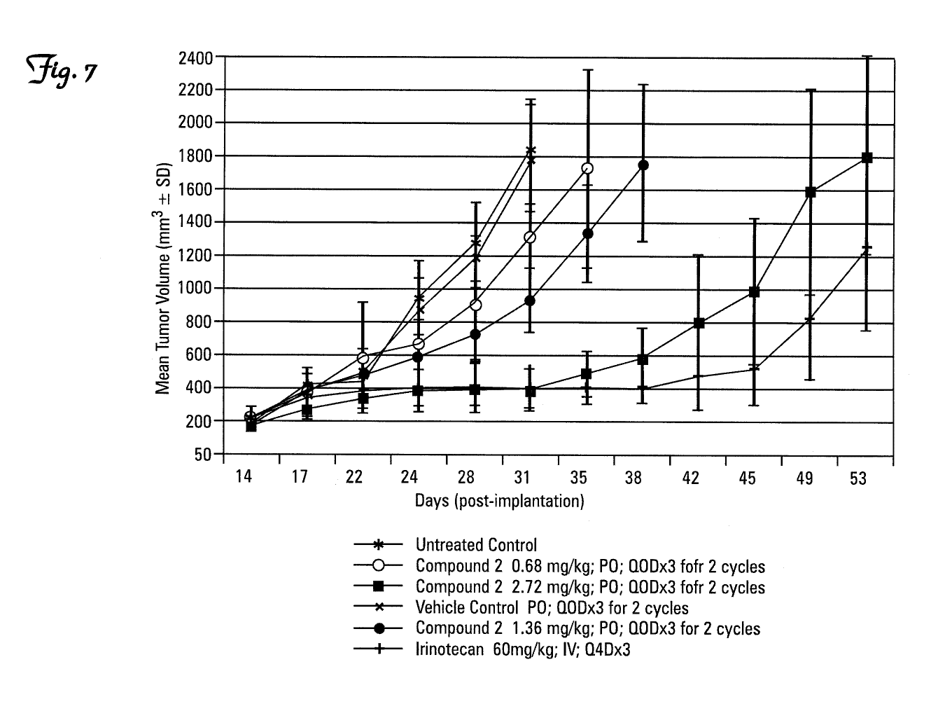

The 1.36 and 2.72 mg/kg/day PO dosages of Compound 2 proved to be effective against the HCT-1 16 human colon tumor xenograft model. Although these dosages were active, irinotecan proved to have slightly superior activity (See FIG. 7).

### Test C In Vitro Primary Pharmacodynamic Studies

The RPMI 8226 (multiple myeloma) human tumor cell line was exposed to Compound 2 (free base) (or simply referred to herein throughout as “Compound 2”) at concentrations covering a 4-log range (0.1 nM -100 nM) with an exposure time of 72 hours and experimental endpoint of cell growth inhibition as determined by a Cell TiterGlo luminescence assay (Promega) for ATP content. At least two independent experiments were conducted. The results were plotted and trend lines were graphed. The IC₅₀ concentration value was found to be 3.4 nM and the IC₉₀ concentration value was found to be 30 nM. As with Compound 2 citrate salt, Compound 2 was shown to be a potent growth inhibitor of these human tumor cells in this cell culture study. Exposure to Compound 2 produced exponential killing of cells in a manner consistent with potent inhibition of a critical molecular target.

### Test D In Vivo Primary Pharmacodynamics

The anti-tumor activity of Compound 2 (free base) was evaluated against a variety of human tumor xenograft models. A summary of the studies, including tumor type, dosing and administration, growth inhibition, and major findings is presented below.

| | | | | | Tumor | Tumor Growth | |
|---|---|---|---|---|---|---|---|
| | | | | | Growth | Delay | |
| Xenograft | Number of | Route of | | Dosage | Inhibition | | Increase in |
| Model/Brief | Animals/ | Administration/ | Compound*/ | (mg/kg/ | T/C (tumor | T-C | Life Span |
| Study Title | Group | Frequency | Dose Schedule | day) | volume) | (Days) | (ILS) |
| Human LOX- | 9 female | IV, qod × 3 for | Untreated | — | — | — | — |
| IMVI Melanoma | nu/nu mice | 2 cycles ^{(a)} | Control | | | | |
| Tumor | per group. | IP, qd × 5 ^{(b)} | Compound 2 ^{(a)} | N/A | N/A | N/A | N/A |
| Xenograft Model | | | Vehicle Control | | | | |
| | | | Compound 2 ^{(a)} | 1 | 89.4% | 2 | 1.1x |
| | | | Compound 2 ^{(a)} | 2 | 66.1% | 25 | 2.8x |
| | | | Compound 2 ^{(a)} | 4 | 39.1% | N/A | N/A |
| | | | Dacarbazine ^{(b)} | 90 | 80.6% | 28 | 2.0x |
| Human DLD-1 | 10 female | IV, qod × 3 for | Untreated | — | — | — | — |
| Colon Tumor | nu/nu mice | 2 cycles ^{(a)} | Control | | | | |
| Xenograft Model | per group. | IV, q4d × 3 ^{(b)} | Compound 2 ^{(a)} | N/A | N/A | N/A | N/A |
| | | | Vehicle Control | | | | |
| | | | Compound 2 ^{(a)} | 1 | 51.3% | 8 | 1.2x |
| | | | Compound 2 ^{(a)} | 2 | N/A | N/A | N/A |
| | | | Compound 2 ^{(a)} | 4 | N/A | N/A | N/A |
| | | | Irinotecan ^{(b)} | 60 | 50.2% | 5 | 1..1x |
| Human HCT-15 | 10 female | IV, qod × 3 for | Untreated | — | — | — | — |
| Colon Tumor | nu/nu mice | 2 cycles ^{(a)} | Control | | | | |
| Xenograft Model | per group. | IV, q4d × 3 ^{(b)} | Compound 2 ^{(a)} | N/A | N/A | N/A | N/A |
| | | | Vehicle Control | | | | |
| | | | Compound 2 ^{(a)} | 1 | 37.1% | 14 | 1.3x |
| | | | Compound 2 ^{(a)} | 2 | 8.6% | 35 | 1.8x |
| | | | Compound 2 ^{(a)} | 2.7 | N/A | N/A | N/A |
| | | | Irinotecan ^{(b)} | 60 | 16.0% | 28 | 1.7x |
| Human NCI- | 9 female | IV, qod × 3 for | Untreated | — | — | — | — |
| H292 Lung | nu/nu mice | 2 cycles ^{(a)} | Control | | | | |
| Tumor | per group. | IV, qod × 3 ^{(b)} | Compound 2 ^{(a)} | N/A | N/A | N/A | N/A |
| Xenograft Model | | | Vehicle Control | | | | |
| | | | Compound 2 ^{(a)} | 1 | 29.4% | 18 | 1.5x |
| | | | Compound 2 ^{(a)} | 1.36 | 25.2% | 21 | 1.6x |
| | | | Compound 2 ^{(a)} | 1.7 | 15.2% | 21 | 1.6x |
| | | | Docetaxel ^{(b)} | 16 | 8.7% | 39 | 2.1x |
| | | | Docetaxel ^{(b)} | 20 | 6.0% | 39 | 2.1x |
| Human H460 | 7 female | IV, qod × 3 for | Untreated | — | — | — | — |
| Non-Small Cell | nu/nu mice | 2 cycles ^{(a)} | Control | | | | |
| Lung Carcinoma | per group. | IV, qod × 3 ^{(b)} | Compound 2 ^{(a)} | N/A | N/A | N/A | N/A |
| Tumor | | | Vehicle Control | | | | |
| Xenograft Model | | IP, qod × 3 ^{(c)} | Compound 2 ^{(a)} | 1.36 | 14.1% | 21 | 1.8x |
| | | | Compound 2 ^{(a)} | 1.36 ^{(a)} | 8.0% | 25 | 1.9x |
| | | | +Docetaxel ^{(b)} | −12 ^{(b)} | | | |
| | | | Compound 2 ^{(a)} | 1.36 ^{(a)} | N/A | N/A | N/A |
| | | | +Cisplatin ^{(c)} | +3.3 ^{(c)} | | | |
| | | | Docetaxel ^{(b)} | 12 | 49.8% | 11 | 1.4x |
| | | | Cisplatin ^{(c)} | 3.3 | 44.5% | 11 | 1.4x |
| Human 786-0 | 10 female | IV, qod × 3 for | Untreated | — | — | — | — |
| Renal Cell | nu/nu mice | 2 cycles ^{(a)} | Control | | | | |
| Tumor | per group. | IV, q4d × 3 ^{(b)} | Compound 2 ^{(a)} | N/A | N/A | N/A | N/A |
| Xenograft Model | | | Vehicle Control | | | | |
| | | | Compound 2 ^{(a)} | 1 | 52.5% | 10 | 1.2x |
| | | | Compound 2 ^{(a)} | 1.36 | 52.2% | 10 | 1.2x |
| | | | Compound 2 ^{(a)} | 1.7 | 25.9% | 17 | 1.4x |
| | | | Irinotecan ^{(b)} | 60 | 49.1% | 17 | 1.4x |
| Human H1299 | 9 female | IV, qod × 3 for | Untreated | — | — | — | — |
| Lung Tumor | nu/nu mice | 2 cycles ^{(a)} | Control | | | | |
| Xenograft Model | per group. | IV, qod × 3 ^{(b)} | Compound 2 ^{(a)} | N/A | N/A | N/A | N/A |
| | | | Vehicle Control | | | | |
| | | | Compound 2 ^{(a)} | 1 | 22.6% | 20 | 1.7x |
| | | | Compound 2 ^{(a)} | 1.36 | 13.2% | 24 | 1.8x |
| | | | Compound 2 ^{(a)} | 1.7 | 8.7% | 34 | 2.1x |
| | | | Docetaxel ^{(b)} | 16 | 35.2% | 11 | 1.4x |
| | | | Docetaxel ^{(b)} | 20 | 20.7% | 17 | 1.6x |
| Human MDA- | 9 female | IV, qod × 3 for | Untreated | — | — | — | — |
| MB-231 Breast | nu/nu mice | 2 cycles ^{(a)} | Control | — | — | — | — |
| Tumor | per group. | IV, qod × 3 ^{(b)} | Compound 2 ^{(a)} | N/A | N/A | N/A | N/A |
| Xenograft Model | | | Vehicle Control | | | | |
| | | | Compound 2 ^{(a)} | 1 | 30.7% | 21 | 1.7x |
| | | | Compound 2 ^{(a)} | 1.36 | 8.6% | >47 | >2.3x |
| | | | Compound 2 ^{(a)} | 1.7 | 17.7% | 35 | 2.0x |
| | | | Docetaxel ^{(b)} | 16 | 4.8% | >47 | >2.3x |
| | | | Docetaxel ^{(b)} | 20 | 4.0% | >47 | >2.3x |
| Human SK- | 10 female | IV, qod × 3 for | Untreated | — | — | — | — |
| MEL-3 | nu/nu mice | 2 cycles ^{(a)} | Control | — | — | — | — |
| Melanoma | per group. | IP, qd × 5 ^{(b)} | Compound 2 ^{(a)} | N/A | N/A | N/A | N/A |
| Tumor | | | Vehicle Control | | | | |
| Xenograft Model | | | Compound 2 ^{(a)} | 1 | 22.0% | 15 | 1.5x |
| | | | Compound 2 ^{(a)} | 1.36 | 13.4% | 35 | 2.3x |
| | | | Compound 2 ^{(a)} | 1.7 | 16.7% | 26 | 1.9x |
| | | | Dacarbazine ^{(b)} | 90 | 88.5% | 0 | 1.0x |
| Human HCT-116 | 8 female | IV, qod × 3 for | Untreated | — | — | — | — |
| Colon Tumor | nu/nu mice | 2 cycles ^{(a)} | Control | | | | |
| Xenograft Model | per group. | | Vehicle Control | — | — | — | — |
| | | | Compound 5 ^{(a)} | 4 | 27.3% | 25 | 1.8x |
| | | | Compound 5 ^{(a)} | 6 | 23.4% | 28 | 1.9x |
| | | | Compound 5 ^{(a)} | 8 | 27.2% | 32 | 2.0x |
| | | | Compound 6 ^{(a)} | 4 | 82.6% | 4 | 1.1x |
| | | | Compound 6 ^{(a)} | 6 | 73.9% | 7 | 1.2x |
| | | | Compound 6 ^{(a)} | 8 | 67.1% | 4 | 1.1x |
| | | | Compound 2 ^{(a)} | 1.7 | 36.4% | 28 | 1.9x |
| Human HT-29 | 8 female | IV, qod × 3 for | Untreated | — | — | — | — |
| Colorectal | nu/nu mice | 2 cycles ^{(a)} | Control | | | | |
| Tumor | per group. | | Vehicle Control | — | — | — | — |
| Xenograft Model | | | Compound 5 ^{(a)} | 6 | N/A | N/A | N/A |
| | | | Compound 5 ^{(a)} | 8 | N/A | N/A | N/A |
| | | | Compound 5 ^{(a)} | 10 | N/A | N/A | N/A |
| | | | Compound 2 ^{(a)} | 1.7 | N/A | N/A | N/A |

IP = intraperitoneal;
IV = intravenous;
PO = per os (oral)
qod × 3 weekly for 2 cycles = every other day for 3 dosages each week for 2 weeks.
q4d × 3 = every fourth day for 3 dosages.
q4d × 5 = every fourth day for 5 dosages.
q3d × 4 = every third day for 4 dosages.
qd × 5 = every day for 5 consecutive dosages.
qod × 5 = every other day for 5 dosages.
^{(a)}, ^{(b)}, ^{(c)}, and ^{(d)}: correlates the route of administration with the compound/dose schedule.

Representative compounds of formula I can be prepared as described in the Examples of international patent application number PCT/US02/36901, which are reproduced below.

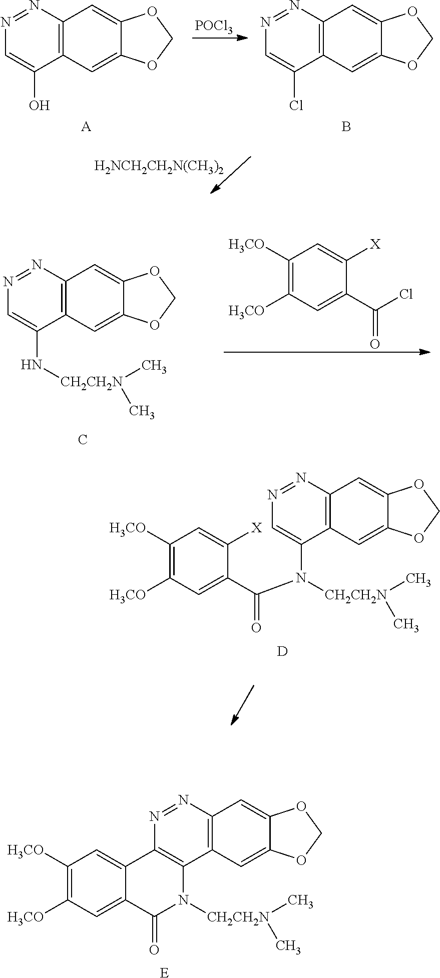

### EXAMPLE 1

11,12-dihydro-2,3-dimethoxy-8,9-methylenedioxy-11-[2-(dimethylamino)ethyl]-5,6,11-triazachrysen-12-one (E). A mixture of 4-N-(2-Dimethylaminoethyl)-N-(2-bromo-4,5-dimethoxybenzoyl)amine-6,7-methylenedioxycinnoline (D, 220 mg, 0.40 mmol), Pd(OAc)₂ (18.0 mg, 0.08 mmol), P(o-tolyl)₃ (48.8 mg, 0.16 mmol), and silver carbonate (225 mg, 0.80 mmol) were heated to reflux in DMF (12 mL) and stirred under nitrogen for 75 minutes. The reaction mixture was cooled to room temperature, diluted with chloroform and filtered though a bed of celite. The solvent was removed under reduced pressure and the resulting residue was chromatographed on silica gel using 95:5 chloroform:methanol to give the title compound (60 mg) in 36% yield; ¹H NMR (CDCl₃) δ 2.42(s, 6H), 3.04(t, 2H, J=7.2 Hz), 4.08(s, 3H), 4.17(s, 3H), 4.64(t, 2H, J=7.2 Hz), 6.25(s, 2H), 7.81(s, 1H), 7.84(s, 1H), 8.07(s, 1H), 8.65(s, 1H); ¹³C NMR (CDCl₃) δ 45.9, 47.4, 56.4, 56.7, 57.7, 99.4, 102.8, 104.3, 106.6, 107.9, 113.7, 119.6, 129.1, 131.0, 134.4, 149.4, 150.2, 151.5, 154.4, 163.1; HRMS calcd. for C₂₂H₂₂O₅N₄H: 423.1668; found 423.1653.

The intermediate 4-N-(2-Dimethylaminoethyl)-N-(2-bromo-4,5-dimethoxybenzoyl)amine-6,7-methylenedioxycinnoline (D) was prepared as follows:

a. 4-N-(2-Dimethylaminoethyl)-N-(2-bromo-4,5-dimethoxybenzoyl)amine-6,7-methylenedioxycinnoline (D). A 2.0M solution of oxalyl chloride in methylene chloride (5 mL, 10.0 mmol) was added to a solution of 2-iodo-4,5-dimethoxybenzoic acid (1.50g, 4.8mmol) in anhydrous methylene chloride (45 mL) and the stirred mixture was refluxed for 2 hours. The mixture was then concentrated to dryness under reduced pressure. To this residue was added a solution of N-(2-Dimethylaminoethyl)-4-amino-6,7-methylenedioxycinnoline (3, 1.0 g, 3.84 mmol), and triethylamine (760 mg 7.52 mmol) in methylene chloride (60 mL) and the resulting mixture was stirred at reflux under nitrogen for 4 hours, then cooled to room temperature; stirring was continued overnight. The reaction mix was washed with a saturated solution of sodium bicarbonate (3×40 mL), dried (anhydrous MgSO₄), and concentrated in vacuo. The crude material was chromatographed over silica using 90:10 chloroform:methanol to give compound D (1.59 g), in 75% yield; ¹H NMR (CDCl₃) δ 2.27(s, 6H), 2.53(m, 2H), 3.43(s, 3H), 3.75(s, 3H), 3.97(m, 1H), 4.44(m, 1H), 6.24(s, 1H), 6.25(s, 1H), 6.43(s, 1H), 7.02(s, 1H), 7.43(s, 1H), 7.68(s, 1H), 9.18(s, 1H); ¹³C NMR (CDCl₃) δ 45.5, 47.1, 55.7, 56.1, 56.7, 82.8, 96.7, 102.9, 105.4, 110.6, 121.9, 123.2, 133.1, 136.0, 144.8, 148.2, 149.9, 150.9, 151.7, 152.4, 169.8; HRMS calcd for C₂₂H₂₃O₅N₄IH: 551.0791; found 551.0795.

b. N-(2-Dimethylaminoethyl)-4-amino-6,7-methylenedioxycinnoline (C). 4-Chloro-6,7-methylenedioxycinnoline (350 mg, 1.7 mmol) and copper powder (100 mg, 1.6 mmol) in N,N-dimethylethylenediamine (3.75 g, 42 6 mmol) were stirred at 105° C. under nitrogen for 3 hours. Excess N,N-dimethylethylenediamine was removed by rotoevaporation, and the residue was dissolved in chloroform (50 mL), and washed with water (3×30 mL), dried (anhydrous MgSO₄), and concentrated in vacuo to give compound C (324 mg) in 74% yield; ¹H NMR (CDCl₃) δ 2.33 (s, 6H), 2.70 (t, 2H), 3.38 (dt, 2H), 6.15 (s, 2H), 7.03 (s, 1H), 7.56 (s, 1H), 8.53 (s, 1H); ¹³C NMR (CDCl₃) δ 39.5, 45.1, 57.0, 94.7, 102.1, 105.3, 112.7, 128.8, 139.8, 147.8, 149.5, 150.7; HRMS calcd for C₁₃H₁₆O₂N₄: 260.1273; found 260.1267.

c. 4-Chloro-6,7-methylenedioxycinnoline (B). 4-Hydroxy-6,7-methylenedioxycinnoline (A, 1.0 g, 5 3 mmol) was added in small portions to a stirred mixture of phosphorus pentachloride (1.4 g, 6.7 mmol) and phosphorus oxychloride (4 mL, 6.6 mmol) at room temperature. The reaction flask was heated to 80° C. for 1 hour, then cooled to room temperature and poured onto 50 g of crushed ice. After neutralization of the solution with solid sodium acetate the precipitate was removed by filtration and recrystallized from ethanol to give 800 mg of 4-chloro-6,7-methylenedioxycinnoline, compound B, in 73% yield; ¹H NMR (CDCl₃) δ 6.25 (s, 2H), 7.39 (s, 1H), 7.73 (s, 1H), 9.14 (s, 1H); ¹³C NMR (CDCl₃) δ 97.8, 102.9, 105.1, 124.2, 133.4, 144.0, 150.0, 152.3, 152.7; HRMS calcd for C₉H_{S}O₂N₂Cl: 208.0040; found 208.0042.

d. 4-Hydroxy-6,7-methylenedioxycinnoline (A). 6′-Amino-3′,4′-(methylenedioxy)acetophenone (2.4 g, 13.4 mmol) in concentrated hydrochloric acid (92 mL) and water (13 mL) was cooled to -5° C. and a diazotized by the dropwise addition of a solution of sodium nitrite (0.925 g, 13 4 mmol) in water (4 mL). After stirring for an additional hour at −5° C. the mixture was transferred to a bath preheated at 75° C. and left to stir at this temperature overnight. The reaction mixture was cooled to 5° C. to complete crystallization of the product in the form of its hydrochloride salt. This material was filtered and then added to 10% aqueous NaOH (100 mL) to generate the free base, which was again filtered and dried under vacuum to yield 2.37 g of the hydroxycinnoline, compound 1, in 93% yield; ¹H NMR (d₆-DMSO) δ 6.21(s, 2H), 6.97 (s, 1H), 7.30 (s, 1H), 7.63 (s, 1H); ¹³C NMR (d₆-DMSO) δ 94.9, 100.29, 103.3, 120.1, 139.7, 139.9, 147.4, 153.5, 169.4; HRMS calcd for C₉H₆O₃N₂: 190.0378; found 190.0372.

### Examples 2-6

The representative compounds of the invention at Examples 2-6 were prepared using the following general procedure from the intermediates prepared in the correspondingly numbered sub-parts a below.

A mixture of the requisite 4-amino-6,7-methylenedioxycinnoline o-iodobenzamide derivative (1.0 mmol equiv.), Pd(OAc)₂ (0.2 mmol equiv.), P(o-tolyl)₃ (0.4 mmol equiv.), and Ag₂CO₃ (2.0 mmol equiv) was heated to reflux in DMF (30 mL per mmol equiv.) with stirring. The reaction mixture was allowed to cool to room temperature, diluted with CHCl₃, and filtered through Celite. The sicciate was extensively washed with 10% CH₃OH in CHCl₃. The filtrate was concentrated in vacuo and the residue chromatographed on silica gel using chloroform:methanol to provide the title compound.

### Example 2

2,3-Dimethoxy-8,9-methylenedioxy-11-[(2-diethylamino)ethyl]-11H-5,6,11-triaza-chrysen-12-one: Prepared from N-(6,7-Methylenedioxycinnolin-4-yl)-N-(N,N-diethylaminoethyl)-2-iodo-4,5-dimethoxybenzamide (578 mg, 1.0 mmol); (18% yield); reaction time 25 min; mp 245-247° C. (dec.); IR (CHCl₃) 1652; ¹H NMR (CDCl₃) δ 1.08 (t, 6H, J=7.0), 2.67 (q, 4H, J=7.0), 3.14 (t, 2H, J=7.1), 4.08 (s, 3H), 4.17 (s, 3H), 4.64 (t, 2H, J=7.1), 6.25 (s, 2H), 7.80 (s, 1H), 7.84 (s, 1H), 8.18 (s, 1H), 8.63 (s, 1H); ¹³C NMR (CDCl₃) δ 11.8, 47.7, 48.0, 51.5, 56.4, 56.6, 99.7, 102.7, 104.3, 106.4, 108.0, 113.7, 119.7, 129.1, 131.1, 134.4, 149.4, 150.3, 151.2, 151.5, 154.4, 163.2; HRMS calcd for C₂₄H₂₆O₅N₄H: 451.1952; found: 451.1960.

### Example 3

2,3-Dimethoxy-8,9-methylenedioxy-11-[(2-dimethylamino)-1-methylethy]-11H-5,6,11-triaza-chrysen-12-one: Prepared from N-(6,7-Methylenedioxycinnolin-4-yl)-N-[2-(N,N-dimethylamino)-1-methylethyl)-2-iodo-4,5-dimethoxybenzamide (100 mg, 0.18 mmol); (28% yield); reaction time 2 h; mp 235-36° C.; IR(KBr) 1659: ¹H NMR (CDCl₃) δ 1.93 (d, 3H, J=8.2), 1.97 (s, 3H), 2.74 (dd, 1H, J=5.8, 13.6), 127 (dd, 1H, J=7.4,12.8), 4.07 (s, 3H), 4.15 (s, 3H), 4.80 (m, 1H), 6.24 (s,2H), 7.74 (s,1H), 7.81 (s,1H), 8.57 (s,1H); ¹³C (CDCl₃) δ 19.4, 45.6, 56.3, 58.6, 63.0, 99.0, 102.6, 104.1, 106.2, 107.9, 114.2, 120.8, 125.6, 128.6, 131.0, 132.5, 132.8, 135.1, 149.2, 150.3, 150.6, 151.3, 154.2, 164.0; HRMS calcd for C₂₃H₂₄N₄O₅H 436.1747; found 436.1832.

### Example 4

2,3-Dimethoxy-8,9-methylenedioxy-11-(2-tetrahydofuranyl)methyl-11H-5,6,11-triazachrysen-12-one: Prepared from N-(6,7-Methylenedioxycinnolin-4-yl)-N-[2-(tetrahydrofuran-2-yl)methyl]-2-iodo-4,5-dimethoxybenzamide (140 mg, 0.25 mmol); (22% yield); reaction time 45 min; mp 300-303° C. (dec.) ; IR (CHCl₃) 1653; ¹H NMR (CDCl₃) δ 1.79 (m, 1H), 2.00 (m, 2H), 2.25 (m, 1H), 3.87 (m, 2H), 4.09 (s, 3H), 4.18 (s, 3H), 4.65 (m, 3H), 6.25 (s, 2H), 7.80 (s, 1H), 7.84 (s, 1H), 8.32 (s, 1H), 8.63 (s, 1H); ¹³C NMR (CDCl₃) δ 25.7, 30.8, 53.0, 56.4, 56.7, 68.4, 77.8, 100.0, 102.7, 104.3, 106.3, 108.0, 114.1, 119.7, 129.1, 131.4, 134.5, 149.5, 150.2, 150.8, 151.4, 154.4, 163.7; HRMS calcd for C₂₃H₂₁O₆N₃: 435.1430; found: 435.1427.

### Example 5

2,3-Dimethoxy-8,9-methylenedioxy-11-[2-(pyrrolidin-1-yl)ethyl]-11H-5,6,11-triaza-chrysen-12-one: Prepared from N-(6,7-Methylenedioxycinnolin-4-yl)-N-[(2-pyrrolidin-1-ypethyl]-2-iodo-4,5-dimethoxybenzamide (150 mg, 0.2 mmol) in 24% yield with a reaction time 30 min; mp 229° C.; IR (KBr) 1644; ¹H NMR (CDCl₃) δ 1.83 (m, 4H), 2.71 (m, 4H), 3.23 (t, 2H, J=7), 4.06 (s, 3H), 4.61 (s, 3H), 4.63 (t, 2H, J=7), 6.23 (s, 2H), 7.74 (s, 1H), 7.80 (s, 1H); ¹³C NMR (CDCl₃) δ 23.7, 54.0, 54.2, 56.3, 56.6, 99.4, 102.7, 104.2, 106.3, 107.7, 113.5, 119.4, 129.0, 134.1, 140.2, 150.2, 151.4, 154.3, 154.3, 163.0; HRMS calcd for C₂₄H₂₄N₄O₅H: 449.1825; found 449.1822.

### Example 6

2,3-Dimethoxy-8,9-methylenedioxy-11-[2-(piperidin-1-yl)ethyl]-11H-5,6,11-triaza-chrysen-12-one: Prepared from N-(6,7-Methylenedioxy-4-cinnolin-4-yl)-N-[2-(piperidin-1-ypethyl]-2-iodo-4,5-dimethoxybenzamide (295 mg, 0.5 mmol); (32.4% yield); reaction time 30 min; mp 294-95° C.; IR (KBr) 1662;¹HNMR (CDCl₃) δ 1.59 (s, 6H), 2.51 (s, 4H), 3.02 (t, 2H, J=6.6), 4.08 (s, 3H), 4.17 (s, 3H), 4.64 (t, 2H, J=6.6), 6.26 (s, 2H), 7.81 (s,1H), 7.85 (s, 1H), 8.36 (s, 1H), 8.65 (s, 1H); ¹³C (CDCl₃) δ 24.3, 26.0, 47.5, 55.0, 56.3, 56.6, 57.4, 99.9, 102.7, 104.2, 106.3, 107.9, 113.7, 119.6, 129.0, 131.1, 134.3, 149.3, 150.2, 151.1, 151.4, 154.3, 163.1; HRMS calcd for C₂₅H₂₆N₄O₅H 463.1981; found 463.1986.

### Examples 2.a-6.a

The intermediate 4-amino-6,7-methylenedioxycinnoline o-iodobenzamide derivatives used in Examples 2-6 were prepared using the following general procedure.

A 2.0M solution of oxalyl chloride in CH₂Cl₂ (1.3 equiv.) was added to a solution of 2-iodo-4,5-dimethoxybenzoic acid (1.0 equiv.) in anhydrous CH₂Cl₂ (≈60 mL per 10 mmol benzoic acid) and the solution stirred at reflux for 3 h. The mixture was allowed to cool and was then concentrated to dryness in vacuo. To the residues was added a solution of requsite 4-amino-6,7-dimethoxyquinoline (1.0 equiv), triethylamine (2 equiv.) in CH₂Cl₂ (≈60 mL per 4 mmol aminoquinoline). The reaction mixture was then stirred at reflux under N₂. The reaction mixture was cooled and washed with sat. NaHCO₃ and extracted with 3% HCl. The aqueous layer was neutralized with 20% NaOH and extracted with CHCl₃, dried (MgSO₄) and evaporated.

### Example 2.a

N-(6,7-Methylenedioxycinuolin-4-yl)-N-(N,N-diethylaminoethyl)-2-iodo-4,5-dimethoxybenzamide: Prepared from N′-(6,7-Methylenedioxycinnolin-4-yl)-N,N-diethylethane-1,2-diamine (640 mg, 2.2 mmol); (87% yield); reaction time 16 h; IR (CHCl₃) 1656; ¹H NMR (CDCl₃) δ 0.92 (t, 6H, J=7.0), 2.50 (q, 4H, J=7.0), 2.80 (t, 2H, J=6.8), 3.39 (s, 3H), 3.71 (s, 3H), 3.94 (m, 1H), 4.41 (m, 1H), 6.21 (d, 2H, J=1.4), 6.39 (s, 1H), 7.01 (s, 1H), 7.39 (s, 1H), 7.64 (s, 1H), 9.11 (s, 1H); ¹³C NMR (CDCl₃) 6 11.6, 46.9, 47.8, 51.1, 55.7, 56.1, 82.9, 96.9, 102.9, 105.5, 110.9, 122.1, 122.9, 133.0, 136.5, 144.9, 148.3, 150.1, 150.9, 151.7, 152.3, 169.8; HRMS calcd for C₂₄H₂₇O₅N₄IH: 579.1105; found: 579.1105.

### Example 3.a

N-(6,7-Methylenedioxycinnolin-4-yl)-N-[2-(N,N-dimethylamino)-1-methylethyl)-2-iodo-4,5-dimethoxybenzamide: Prepared from N-(6,7-difluorocinnolin-4-yl)-N¹,N¹-dimethylpropane-1,2-diamine (240 mg, 0.87 mmol); (83% yield); reaction time 16 h, mp 110-111° C.; ¹H NMR (CDCl₃) was a mixture of atropisomers 6 isomer #1 1.03-1.36 (m, 3H), 2.21-2.37 (m, 6H), 2.74-3.07 (m, 1H), 3.43-3.65 (m, 6H), 3.84-3.91 (m, 1H), 5.15 (m, 1H), 6.18 (s, 2H), 6.59 (s, 1H), 6.91 (s, 1H), 7.56 (s, 1H), 8.04 (s, 1H), 9.34 (s, 1H) isomer #2 1.03-1.36 (m, 3H), 2.31-2.37 (m, 6H), 2.74-3.07 (m, 1H), 3.43-3.65 (m, 6H), 3.84-3.91 (m, 1H), 5.15 (m, 1H), 6.18 (s, 2H), 6.59 (s, 1H), 6.91 (s, 1H), 7.56 (s, 1H), 8.04 (s, 1H), 9.34 (s, 1H); HRMS calcd for C₂₃H₂₅O₅N₄IH: 565.0870; found: 565.0926.

### Example 4.a

N-(6,7-Methylenedioxycinnolin-4-yl)-N-[2-(tetrahydrofuran-2-yl)methyl]-2-iodo-4,5-dimethoxybenzamide: Prepared from 2-[[[N-(6,7-Methylenedioxycinnolin-4-yl)]amino]methyl]tetrahydrofuran (400 mg, 1.5 mmol); (34% yield); reaction time 16 h; IR (CHCl₃) 1654; ¹H NMR, a mixture of atropisomers, (CDCl₃) δ isomer #1 1.94 (m, 4H), 3.70 (m, 4H), 3.73 (s, 3H), 3.94 (s, 3H), 4.34 (m, 1H) 6.23 (s, 2H), 7.00 (s, 1H), 7.40 (s, 1H), 7.70 (s, 1H), 9.31 (s, 1H), isomer #2 1.94 (m, 4H), 3.70 (m, 4H), 3.73 (s, 3H), 3.94 (s, 3H), 4.34 (m, 1H) 6.46 (s, 2H), 7.36 (s, H), 7.49 (s, 1H), 7.65 (s, 1H), 9.17 (s, 1H); HRMS calcd for C₂₃H₂₂O₆N₃IH: 564.0632; found: 564.0650.

### Example 5.a

N-(6,7-Methylenedioxycinnolin-4-yl)-N-[(2-pyrrolidin-l-yl)ethyl]-2-iodo-4,5-dimethoxybenzamide: Prepared from 1-[2-[N-(6,7-Methylenedioxycinnolin-4-yl)]amino]ethylpyrrolidine (400 mg, 0.4 mmol) in 42% yield with a reaction time 4 h at 50° C. from the acid chloride prepared using 4.1 mmol of oxalyl chloride and 1.6 mmol of 2-iodo-4,5-dimethoxybenzoic acid. Compound 8f had: IR (KBr) 1655; ¹H NMR (CDCl₃) δ 1.60 (m, 4H), 2.40 (m, 4H), 2.67 (m, 2H), 3.28 (s, 3H), 3.60 (s, 3H), 4.32 (m, 1H), 6.11 (d, 2H, J=2.2), 6.32 (s, 1H), 6.91 (s, 1H), 7.37 (s, 1H), 7.50 (s 1H), 9.04 (s, 1H); ¹³C NMR (CDCl₃) δ 23.6, 29.7, 47.6, 52.9, 53.9, 55.7, 56.0, 56.4, 82.8, 96.7, 102.9, 105.4, 110.6, 121.9, 123.1, 132.8, 135.9, 144.7, 148.2, 149.9, 150.9, 151.7, 152.4, 169.9.

### Example 6.a

N-(6,7-Methylenedioxy-4-cinnolin-4-yl)-N-[2-(piperidin-1-yl)ethyl]-2-iodo-4,5-dimethoxybenzamide: Prepared from 1-[2-[N-(6,7-Methylenedioxycinnolin-4-yl)]amino]ethylpiperidine (500 mg, 1.66 mmol); (85.4% yield); reaction time overnight at 50° C. mp 93-94° C.; IR (KBr) 1655; ¹HNMR (CDCl₃) δ 1.43 (m, 6H), 2.35 (m, 4H), 2.50-2.71 (m, 2H), 3.43 (s, 3H), 3.73 (s, 3H), 3.78-3.93 (m, 1H), 4.32.4.42 (m, 1H), 6.22 (d, 2H, J=1.6), 6.42 (s, 1H), 7.02 (s, 1H), 7.47 (s, 1H), 7.66 (s, 1H), 9.19 (s, 1H); ¹³C (CDCl₃) δ 24.3, 25.9, 46.0, 46.4, 54.5, 55.6, 56.0, 56.4, 82.9, 97.0, 102.8, 105.3, 110.8, 122.0, 113.7, 123.2, 133.1, 136.3, 145.0, 148.2, 149.9, 150.8, 151.6, 152.1, 169.8 HRMS calcd for C₂₃H₂₅IN₄O₅H: 591.1105; found 591.1108.

### Examples 2.b-6.b

The intermediate 4-amino-6,7-dimethoxyquinoline derivatives used in Examples 2.a-6.a. were prepared using the following general procedure.

The appropriate primary amine (1.0 mol equiv.) added with stirring to 4-Chloro-6,7-methylenedioxycinnoline (see Example 1 above). The reaction was then allowed to stir at 100° C. for several hours, and the phenol removed by Kugelrohr distillation under reduced pressure. The residue was partitioned between CHCl₃ and 10% NaOH. The aqueous layer was repeatedly separated with CHCl₃. All of the CHCl₃ solutions (initial partition and extracts) were combined and dried (MgSO₄).

### Example 2.b

### N′-(6,7-Methylenedioxycinnolin-4-yl)-N,N-diethylethane-1,2-diamine

Prepared from 4-Chloro-6,7-methylenedioxycinnoline (1.0 g, 4.8 mmol); (70% yield); reaction time 3 h; mp 230-232° C.; ¹H NMR (CDCl₃) δ 1.10 (t, 6H, J=7.2), 2.63 (q, 4H, J=7.2), 2.84 (t, 2H, J=5.7), 3.35 (q, 2H, J=5.7), 5.78 (br, 1H), 6.15 (s, 2H), 6.96 (s, 1H), 7.57 (s, 1H), 8.52 (s, 1H); ¹³C NMR (CDCl₃) δ 12.2, 39.5, 46.6, 50.8, 94.4, 102.0, 105.4, 112.8, 129.0, 139.8, 147.8, 149.5, 150.7; HRMS calcd for C₁₅H₂₀O₂N₄: 288.1586; found: 288.1575.

### Example 3.b

N-(6,7-difluorocinnolin-4-yl)-N¹,N¹-dimethylpropane-1,2-diamine: Prepared from 4-Chloro-6,7-methylenedioxycinnoline (0.52 g, 2.5 mmol); (42% yield), reaction time 4 h, mp 196-197° C.; ¹H NMR (CD₃OD) δ 1.31 (d, 3H, J=6.6), 2.33 (s, 6H), 2.45 (dd, 1H, J=5.4, 12.8), 2.74 (dd, 1H, J=8.2, 12.6), 4.12 (dd, 1H, J=5.8, 13.8), 6.19 (s, 2H), 7.32 (s, 1H), 7.56 (s, 1H), 8.51 (s, 1H); ¹³C NMR (CD₃OD) δ 17.1, 44.0, 45.3, 63.5, 95.1, 101.6, 102.0, 112.6, 126.7, 140.8, 149.3, 151.2; HRMS calcd for C₁₄H₁₈O₂N₄: 274.1430; found: 274.1429.

### Example 4.b

### 2-[[[N-(6,7-Methylenedioxycinnolin-4-yl)]amino]methyl]tetrahydrofuran

prepared from 4-Chloro-6,7-methylenedioxycinnoline (500 mg, 2.4 mmol); (78% yield); reaction time 2 h; mp 196-198° C.; ¹H NMR (CDCl₃) δ 1.74 (m, 1H), 2.11 (m, 3H), 3.30 (m, 1H), 3.58 (m, 1H), 3.92 (m, 2H), 4.29 (m, 1H), 5.22 (br, 1H), 6.12 (s, 2H), 6.98 (s, 1H), 7.52 (s, 1H), 8.54 (s, 1H); ¹³C NMR (CDCl₃) δ 25.9, 29.2, 46.9, 68.4, 76.9, 94.4, 102.2, 105.2, 112.8, 128.7, 139.8, 147.9, 149.6, 150.8; HRMS calcd for C₁₄H₁₅O₃N₃: 273.1130; found: 273.1130.

### Example 5.b

1-[2-[N-(6,7-Methylenedioxycinnolin-4-yl)]amino]ethylpyrrolidine: Prepared from 4-Chloro-6,7-methylenedioxycinnoline (750 mg, 3.5 mmol), 1-(2-aminoethyl)pyrrolidine (3 ml) and copper powder (300 mg) in 75% yield; reaction time 18 h at 90° C.; mp 215° C. (dec); ¹H NMR (CDCl₃) δ 1.85 (m, 4H), 2.63 (m, 4H), 2.90 (t, 2H, J=6), 3.42 (t, 2H, J=6), 5.63 (s, 1H), 6.14 (s, 2H), 7.04 (s, 1H), 7.57 (s, 1H), 8.53 (s, 1H); ¹³C NMR (DMSO-d₆) δ 23.9, 42.0, 54.5, 54.7, 97.0, 102.9, 104.4, 112.7, 126.8, 140.8, 149.3, 151.0; HRMS calcd for C₁₅H₁₈N₄O₂: 293.1590; found 293.1579.

### Example 6.b

1-[2-[N-(6,7-Methylenedioxycinnolin-4-yl)]amino]ethylpiperidine:—Prepared from 4-Chloro-6,7-methylenedioxycinnoline (1.04 g, 5.0 mmol); (37% yield); reaction time 2 h; mp 238-239 C; ¹H NMR (CD₃OD) δ 1.56 (d, 2H, J=5.2), 1.70 (d, 2H, J=4.6), 2.87 (t, 2H, J=7), 3.65 (t, 2H, J=6.6), 6.20 (s, 2H), 7.32 (s, 1H), 7.43 (s, 1H), 8.46 (s, 1H); ¹³C(CD₃OD) δ 23.1, 24.7, 38.5, 53.6, 56.1, 94.7, 101.7, 102.1, 112.4, 126.6, 141.1, 14.7, 149.4, 151.2 (CDCl₃);HRMS calcd for C₁₆H₂₀N₄O₂H: 300.1586; found 300.1586.

### Examples 7-12

The representative compounds of the invention at Examples 7-12 were prepared using the following general procedure from the intermediates prepared in the correspondingly numbered sub-parts a below.

A mixture of the requsite 4-amino-6,7-methylenedioxyquinoline o-iodobenzamide derivative (1.0 mmol equiv.), Pd(OAc)₂ (0.2 mmol equiv.), P(o-tolyl)₃ (0 4 mmol equiv.), and Ag₂CO₃ (2.0 mmol equiv) was heated to reflux in DMF (30 mL per mmol equiv.) with stirring. The reaction mixture was allowed to cool to room temperature, diluted with CHCl₃, and filtered through Celite. The sicciate was extensively washed with 10% CH₃OH in CHCl₃. The filtrate was concentrated in vacuo and the residue chromatographed on silica gel using chloroform:methanol.

### Example 7

8,9-Dimethoxy-2,3-methylenedioxy-5-[2-(N,N-dimethylamino)ethyl]-5H-dibenzo[c,h]1,6-naphthyridin-6-one. Prepared from N-(6,7-Methylenedioxyquinolin-4-yl)-N-(N,N-dimethylaminoethyl)-2-iodo-4,5-dimethoxybenzamide; (41% yield); reaction time 25 min; mp 283-285° C. (dec.); IR (CHCl₃) 1653; ¹H NMR (CDCl₃) δ 2.33 (s, 6H), 3.04 (t, 2H, J=7.2), 4.07 (s, 3H), 4.14 (s, 3H), 4.64 (t, 2H, J=7.2), 6.18 (s, 2H), 7.47 (s, 1H), 7.68 (s, 1H), 7.89 (s, 2H), 9.37 (s, 1H); ¹³C NMR (CDCl₃) δ 45.9, 49.2, 56.3, 56.3, 57.9, 101.2, 102.0, 102.3, 107.1, 108.8, 111.7, 114.8, 119.3, 127.6, 140.9, 143.5, 147.3, 147.7, 149.9, 150.3, 154.2, 164.1; HRMS calcd for C₂₃H₂₃N₃O₅H: 422.1716; found 422.1710.

### Example 8

8,9-Dimethoxy-2,3-methylenedioxy-5-[2-(N,N-dimethylamino)-1-methylethyl]-5H-dibenzo[c,h]1,6-naphthyridin-6-one: Prepared from N-(6,7-Methylenedioxyquinolin-4-yl)-N-[2-(N,N-dimethylamino)-1-methylethyl)-2-iodo-4,5-dimethoxybenzamide; (30.4% yield); reaction time 30 min; mp 186-187° C.; IR (KBr) 1649; ¹H NMR (CDCl₃); δ 1.95-1.98 (m, 9H), 2.77 (dd, 1H, J=12.0, 8.0), 3.21 (dd, 1H, J=12.0, 8.0), 4.06 (s, 3H), 4.13 (s, 3H), 4.84-4.92 (m, 1H), 6.17 (s, 2H), 7.46 (s, 1H), 7.66 (s, 1H), 7.77 (s, 1H), 7.87 (s, 1H), 9.35 (s, 1H); ¹³C NMR (CDCl₃) δ 19.7, 45.5, 56.2, 56.3, 59.5, 63.1, 100.9, 101.9, 102.1, 107.0, 108.7, 112.4, 115.2, 120.5, 127.3, 142.6, 143.3, 147.0, 147.3, 149.9, 150.1, 154.0, 164.9; HRMS calcd for C₂₄H₂₅N₃O₅H: 436.1794; found 436.1863.

### Example 9

8,9-Dimethoxy-2,3-methylenedioxy-5-[2-(pyrrolidin-1-yl)ethyl]-5H-dibenzo[c,h]1,6-naphthyridin-6-one: Prepared from N-(6,7-Methylenedioxyquinolin-4-yl)-N-[(2-pyrrolidin-1-yl)ethyl]-2-iodo-4,5-dimethoxybenzamide; (36% yield); reaction time 30 min; mp 255-257° C. (dec.); IR (CHCl₃) 1653; ¹H NMR (CDCl₃) δ 1.79 (m, 4H), 2.64 (m, 4H), 3.20 (t, 2H, J=7.1), 4.07 (s, 3H), 4.14 (s, 3H), 4.69 (t, 2H, J=7.1), 6.18 (s, 2H), 7.46 (s, 1H), 7.68 (s, 1H), 7.89 (s, 1H), 7.95 (s, 1H), 9.37 (s, 1H); ¹³C NMR (CDCl₃) δ 23.7, 49.6, 54.3, 56.3, 56.4, 56.4, 101.3, 102.0, 102.3, 107.0, 108.7, 111.7, 114.8, 119.3, 127.7, 140.9, 143.4, 147.3, 147.8, 150.0, 150.3, 154.2, 164.2; HRMS calcd for C₂₅H₂₅N₃O₅H: 448.1872; found 448.1872.

### Example 10

8,9-Dimethoxy-2,3-methylenedioxy-5-[2-(4-methylpiperazin-1-ypethyl]-5H-dibenzo[c,h]1,6-naphthyridin-6-one: Prepared from N-(6,7-Methylenedioxyquinolin-4-yl)-N-[2-(4-methyl-1-piperazinyl)ethyl]-2-iodo-4,5-dimethoxybenzamide; (18% yield); reaction time 25 min; mp 244-246° C.; IR (CHCl₃) 1651; ¹H NMR (CDCl₃) δ 2.27 (s, 3H), 2.51 (m, 8H), 2.95 (t, 2H, J=6.2), 4.07 (s, 3H), 4.15 (s, 3H), 4.69 (t, 2H, J=6.2), 6.19 (s, 2H), 7.48 (s, 1H), 7.70 (s, 1H), 7.91 (s, 2H), 7.92 (s, 1H), 9.39 (s, 1H); ¹³C NMR (CDCl₃) δ 29.8, 45.9, 48.6, 53.0, 55.0, 56.4, 56.4, 101.2, 102.0, 102.2, 107.1, 108.9, 112.0, 115.0, 119.5, 127.6, 141.2, 143.4, 147.4, 147.2, 150.0, 150.3, 154.1, 164.4; HRMS calcd for C₂₆H₂₈N₄O₅H: 477.2138; found 477.2139.

### Example 11

8,9-Dimethoxy-2,3-methylenedioxy-5-[3-(N,N-dimethylamino)propyl]-5H-dibenzo[c,h]1,6-naphthyridin-6-one): Prepared from N-(6,7-Methylenedioxyquinolin-4-yl)-N-[3-(N,N-dimethylamino)propyl]-2-iodo-4,5-dimethoxybenzamide; (45% yield); reaction time 30 min; mp 262-264° C. (dec.); IR (CHCl₃) 1648; ¹H NMR (CDCl₃) δ 2.29 (m, 8H), 2.45 (m, 2H), 4.07 (s, 3H), 4.14 (s, 3H), 4.53 (t, 2H, J=7.4), 6.19 (s, 2H), 7.48 (s, 1H), 7.65 (s, 1H), 7.69 )s, 1H), 7.90 (s, 1H), 9.40 (s, 1H); ¹³C NMR (CDCl₃) δ 26.9, 45.3, 49.2, 56.3, 56.4, 56.9, 100.8, 101.9, 102.3, 107.1, 108.7, 111.6, 114.9, 119.4, 127.5, 141.0, 143.6, 147.2, 147.7, 149.9, 150.3, 154.1, 164.1; HRMS calcd for C₂₄H₂₅N₃O₅H: 436.1872; found 436.1878.

### Example 12

8,9-Dimethoxy-2,3-methylenedioxy-5-(2-tetrahydofuranyl)methyl-5H-dibenzo[c,h]1,6-naphthyridin-6-one: Prepared from N-(6,7-Methylenedioxyquinolin-4-yl)-N-[2-(tetrahydrofuran-2-yl)methyl]-2-iodo-4,5-dimethoxybenzamide; (22% yield); reaction time 30 min; mp 270-273° C.; IR (CHCl₃) 1648; ¹H NMR (CDCl₃) δ 1.87 (m, 4H), 3.72 (m, 2H), 4.07 (s, 3H), 4.14 (s, 3H), 4.68 (m, 3H), 6.18 (s, 2H), 7.48 (s, 1H), 7.69 (s, 1H), 7.90 (s, 1H), 8.04 (s, 1H), 9.39 (s, 1H); ¹³C NMR (CDCl₃) δ 25.6, 30.3, 54.7, 56.3, 56.4, 68.1, 77.3, 101.7, 102.2, 102.3, 107.0, 109.0, 112.1, 115.2, 119.5, 127.7, 141.2, 143.5, 147.2, 147.4, 149.9, 150.3, 154.2, 164.6; HRMS calcd for C₂₄H₂₂N₂O₆H 435.1556; found 435.1566.

### Examples 7.a-12.a

The intermediate 4-amino-6,7-methylenedioxyquinoline o-iodobenzamide derivatives used in Examples 7-12 were prepared using the following general procedure.

A 2.0M solution of oxalyl chloride in CH₂Cl₂ (1.3 equiv.) was added to a solution of 2-iodo-5,6-dimethoxybenzoic acid (1.0 equiv.) in anhydrous CH₂Cl₂ 60 mL per 10 mmol benzoic acid) and the solution stirred at reflux for 3 h. The mixture was allowed to cool and was then concentrated to dryness in vacuo. To the residue was added a solution of appropriate 4-amino-6,7-dimethoxyquinoline (1.0 equiv), triethylamine (2 equiv.) in CH₂Cl₂ (≈60 mL per 4 mmol aminoquinoline). The reaction mixture was then stirred at reflux under N₂. . In the case of those derivatives that have an alkylamine incorporated in their structure, the residue was partitioned between CHCl₃ and 10% NaOH. The aqueous layer was repeatedly separated with CHCl₃. All of the CHCl₃ solutions (initial partition and extracts) were combined and dried (MgSO₄). The aqueous layer was neutralized with 20% NaOH and extracted with CHCl₃, dried (MgSO₄) and evaporated.

### Example 7.a

N-(6,7-Methylenedioxyquinolin-4-yl)-N-(N,N-dimethylaminoethyl)-2-iodo-4,5-dimethoxybenzamide. Prepared from N′-(6,7-Methylenedioxyquinolin-4-yl)-N,N-dimethylethane-1,2-diamine (1.0 g, 3.84 mmol) in 71% yield with a reaction time of 3 h, from the acid chloride prepared using 10 mmol of oxalyl chloride and 4 8 mmol of 2-iodo-5,6-dimethoxybenzoic acid. Compound 7a had: IR (CHCl₃) 1652; ¹H NMR (CDCl₃) δ 2.74 (s, 6H), 2.66 (t, 2.H, J=7.0), 3.33 (s, 3H), 3.74 (s, 3H), 3.96 (m, 1H), 4.49, (m, 1H), 6.15 (s, 2H), 6.41 (s, 1H), 7.03 (s, 1H), 7.34 (d, 1H, J=4.8), 7.37 (s, 1H), 7.44 (s, 1H), 8.56 (d, 1H, J=4.8); ¹³C NMR (CDCl₃) δ 45.7, 46.9, 55.5, 56.1, 56.6, 82.7, 98.5, 102.2, 106.7, 110.2, 120.2, 121.5, 122.9, 121.5, 122.9, 133.8, 145.9, 148.0, 148.3, 148.5, 149.0, 149.6, 151.0, 170.0; HRMS calcd for C₂₃H₂₄IN₃O₅H: 550.0839; found 550.0823.

### Example 8.a

N-(6,7-Methylenedioxyquinolin-4-yl)-N-[2-(N,N-dimethylamino)-1-methylethyl)-2-iodo-4,5-dimethoxybenzamide. Prepared from N′-(6,7-Methylenedioxyquinolin-4-yl)-N,N-dimethylpropane-1,2-diamine (273 mg, 1.0 mol) in 60.4% yield with a reaction time of 12 h, from the acid chloride prepared using 4 8 mmol of oxalyl chloride and 1.2 mmol of 2-iodo-5,6-dimethoxybenzoic acid. Compound 7b had: mp 82-84° C.; IR (KBr) 1648, 3415; HRMS calcd for C₂₄H₂₆1N₃O₅H 564.0917; found 564.0997

### Example 9.a

N-(6,7-Methylenedioxyquinolin-4-yl)-N-[(2-pyrrolidin-1-yl)ethyl]-2-iodo-4,5-dimethoxybenzamide. Prepared from 1-[2-[N-(6,7-Methylenedioxyquinolin-4-yl)amino]amino]ethylpyrrolidine (285 mg, 1 0 mmol), in 87% yield with a reaction time of 12 h, from the acid chloride prepared using 4 mmol of oxalyl chloride and 1.36 mmol of 2-iodo-5,6-dimethoxybenzoic acid. Compound 7c had: IR (CHCl₃) 1650; ¹H NMR (CDCl₃) δ 1.78 (m, 4H), 2.22 (m, 1H), 2.59 (m, 3H), 2.83 (t, 2H, J=6.6), 3.33 (s, 3H), 3.74 (s, 3H), 3.96 (d, 1H, J=4), 4.54 (m, 1H), 6.15 (s, 1H), 6.42 (s, 1H), 7.03 (s, 1H), 7.34 (d, 1H, J=4.8), 7.36 (s, 1H), 7.44 (s, 1H), 8.55 (d, 1H, J=4.8); ¹³C NMR (CDCl₃) δ 23.7, 47.7, 52.9, 54.1, 55.5, 56.1, 82.7, 98.4, 102.2, 106.7, 106.7, 120.1, 121.5, 122.9, 133.7, 145.9, 148.0, 148.3, 148.4, 149.0, 149.6, 151.0, 170.0; HRMS calcd for C₂₅H₂₆IN₃O₅H: 576.0995; found 576.1003.

### Example 10.a

N-(6,7-Methylenedioxyquinolin-4-yl)-N-[2-(4-methyl-l-piperazinyl)ethyl]-2-iodo-4,5-dimethoxybenzamide. Prepared from 1-[24N-(6,7-Methylenedioxyquinolin-4-yl)]amino]ethyl-4-methylpiperazine (290 mg, 0.9 mmol) in 50% yield with a reaction time of 12 h, from the acid chloride prepared using 4.0 mmol of oxalyl chloride and 1.8 mmol of 2-iodo-5,6-dimethoxybenzoic acid. Compound 7d had: IR (CHCl₃) 1649; ¹H NMR (CDCl₃) δ 2.29 (s, 3H), 2.51 (m, 10H), 3.35 (s, 3H), 3.75 (s, 3H), 3.95 (m, 1H), 4.46 (m, 1H), 6.15 (s, 1H), 6.42 (s, 1H), 7.03 (s, 1H), 7.35 (d, 1H, J=4.6), 7.36 (s, 1H), 7.48 (s, 1H), 8.57 (d, 1H, J=4.6); ¹³C NMR (CDCl₃) δ 46.0, 46.2, 53.1, 55.2, 55.5, 55.5, 56.0, 82.7, 98.7, 102.2, 106.7, 110.4, 120.3, 121.6, 123.0, 133.7, 146.0, 148.0, 148.4, 148.4, 148.9, 149.6, 151.0, 170.0; HRMS calcd for C₂₆H₂₉IN₄O₅H: 605.1261; found 605.1261.

### Example 11.a

N-(6,7-Methylenedioxyquinolin-4-yl)-N-[3-(N,N-dimethylamino)propyl]-2-iodo-4,5-dimethoxybenzamide. Prepared from N′-(6,7-Methylenedioxyquinolin-4-yl)-N,N-dimethylpropane-1,3-diamine (273 mg, 1 0 mmol), in 79% yield with a reaction time of 12 h, from the acid chloride prepared using 4.0 mmol of oxalyl chloride and 1.36 mmol of 2-iodo-5,6-dimethoxybenzoic acid. Compound 7e had: IR (CHCl₃) 1650; ¹H NMR (CDCl₃) δ 1.93 (m, 1H), 2.16 (m, 1H), 2.34 (s, 6H), 2.58 (m, 1H), 3.31 (s, 3H), 3.47 (m, 1H), 3.75 (s, 3H), 3.95 (m, 1H,), 4.55, (m, 1H), 6.16 (s, 1H), 6.39 (s, 1H), 7.04 (s, 1H), 7.28 (d, 1H, J=5.0), 7.31 (s, 1H), 7.38 (s, 1H), 8.56 (d, 1 h, J=5.0); ¹³C NMR (CDCl₃) δ 25.8, 45.1, 47.2, 55.5, 56.1, 26.9, 82.7, 98.1, 102.3, 107.0, 110.1, 120.1, 121.5, 122.5, 133.5, 145.5, 148.1, 148.4, 148.6, 149.2, 149.7, 151.1, 170.1; HRMS calcd for C₂₄H₂₆IN₃O₅H: 564.0995; found 564.0990.

### Example 12.a

N-(6,7-Methylenedioxyquinolin-4-yl)-N-[2-(tetrahydrofuran-2-yl)methyl]-2-iodo-4,5-dimethoxybenzamide. Prepared from 2-[[[N-(6,7-Methylenedioxyquinolin-4-yl)]amino]methyl]tetrahydrofuran (272 mg, 1.0 mol) in 36% yield with a reaction time of 16 h, from the acid chloride prepared using 4.0 mmol of oxalyl chloride and 1.36 mmol of 2-iodo-5,6-dimethoxybenzoic acid. Compound 7g had: IR (CHCl₃) 1652; HRMS calcd for C₂₄H₂₃N₂O₆IH: 563.0679; found 563.0703.

### Examples 7.b-12.b

The intermediate 4-amino-6,7-dimethoxyquinoline derivatives used in Examples 7.a-12.a. were prepared using the following general procedure.

4-Chloro-6,7-methylenedioxyquinoline was stirred in refluxing phenol (5.5 mol equiv.) for 2.5 h. The temperature was lowered to 100° C. and the primary amine (1.0 mol equiv.) added with stirring. The reaction was then allowed to stir at 100° C. for several hours, and the phenol removed by Kugelrohr distillation under reduced pressure. In the case of those derivatives that have an alkylamine incorporated in their structure, the residue was partitioned between CHCl₃ and 10% NaOH. The aqueous layer was repeatedly separated with CHCl₃. All of the CHCl₃ solutions (initial partition and extracts) were combined and dried (MgSO₄). Other 4-amino-6,7-methylenedioxyquinoline derivatives were purified by column chromatography.

### Example 7.b

N′-(6,7-Methylenedioxyquinolin-4-yl)-N,N-dimethylethane-1,2-diamine was prepared from N,N-dimethylethylenediamine (2.55 g, 29 mmol) in 54% yield with a reaction time of 24 h. Compound 6a had: mp 193-194° C.; ¹H NMR (CDCl₃) δ 2.32 (s, 6H), 2.70 (t, 2H, J=6.6), 3.29 (m, 2H), 5.62 (br, 1H), 6.10 (s, 2H), 6.36 (d, 1H, J=5.3), 7.10 (s, 1H), 7.34 (s, 1H), 8.40 (d, 1H, J=5.3); ¹³C NMR (CDCl₃) δ 40.1, 45.2, 57.2, 96.3, 98.9, 101.6, 106.5, 114.4, 145.2, 146.8, 148.9, 149.7, 150.1; HRMS calcd for C₁₄H₁₇N₃O₂: 260.1399; found 260.1377.

### Example 8.b

N′-(6,7-Methylenedioxyquinolin-4-yl)-N,N-dimethylpropane-1,2-diamine was prepared from 2-methyl-2-(N,N-dimethylamino)ethylamine (2.55 g, 29 mmol) from in 30.7% yield with a reaction time of 24 h. Compound 6b had: mp 71-72° C.; ¹H NMR (CD₃OD); δ 1.26 (d, 3H, J=5.6), 3.22 (s, 6H), 2.41 (dd, 1H, J=6.2, 12), 2.65 (dd, 1H, J=5.8, 12.2), 3.82-3.86 (m, 1H), 6.16 (s, 2H), 6.46 (d, 1H, J=5.8), 7.16 (s, 1H), 7.45 s,1H), 8.20 (d, 1H, J=6.0); ¹³C NMR δ 17.1, 44.0, 45.4, 63.6, 96.6, 97.3, 101.3, 101.8, 113.9, 144.8, 146.3, 146.8, 149.7, 150.0; HRMS calcd for C₁₅H₁₉N₃O₂H: 273.1484; found 273.1477.

### Example 9.b

1-[2-[N-(6,7-Methylenedioxyquinolin-4-yl)]amino]ethylpyrrolidine was prepared from 1-(2-aminoethyl)pyrrolidine (1.14 g, 10.0 mmol) in 31% yield with a reaction time of 20 h. Compound 6c had: mp 179-182° C.; ¹H NMR (CDCl₃) δ 1.83 (m, 4H), 2.60 (m, 4H), 2.87 (t, 2H, J=5.9), 3.33 (m, 2H), 5.58 (br, 1H), 6.08 (s, 2H), 6.34 (d, 1H, J=5.1), 7.08 (s, 1H), 7.31 (s, 1H), 8.40 (d, 1H, J=5.1); ¹³C NMR (CDCl₃) δ 23.7, 41.4, 53.9, 54.0, 96.3, 98.9, 101.6, 106.6, 114.4, 146.4, 146.7, 149.1, 149.6, 150.0; HRMS calcd for C₁₆H₁₉N₃O₂: 285.1477; found 285.1468.

### Example 10.b

1-[2-[N-(6,7-Methylenedioxyquinolin-4-yl)]amino]ethyl-4-methylpiperazine was prepared from 2-(4-methylpiperidin-1-yl)ethylamine (1.43 g, 10 0 mmol) in 20% yield with a reaction time of 24 h. Compound 6d had: mp 159-161° C.; ¹H NMR (CDCl₃) δ 2.34 (s, 3H), 2.54 (m, 10H), 2.80 (t, 2H, J=5.9), 5.62 (br, 1H), 6.11 (s, 2H), 6.38 (d, 1H, J=5.2), 7.05 (s, 1H), 7.33 (s, 1H), 8.41 (d, 1H, J=5.2); ¹³C NMR (CDCl₃) δ 39.1, 46.2, 52.7, 55.4, 55.7, 96.0, 99.0, 101.6, 106.6, 114.3, 146.8, 146.8, 149.0, 149.5, 150.0; HRMS calcd for C₁₇H₂₂N₄O₂: 314.1743; found 314.1738.

### Example 11.b

N′-(6,7-Methylenedioxyquinolin-4-yl)-N,N-dimethylpropane-1,3-diamine

was prepared from N,N-dimethyl-1,3-diaminopropane (1.0 g, 10.0 mmol) in 25% yield with a reaction time of 20 h. Compound 6e had: mp 178-181° C.; ¹H NMR (CDCl₃) δ 1.92 (m, 2H), 2.39 (s, 6H), 2.58 (t, 2H, J=5.5), 3.39 (m, 2H), 6.08 (s, 2H), 6.29 (d, 1H, J=5.6), 6.95 (s, 1H), 7.31 (s, 1H), 7.52 (br s, 1H), 8.37 (d, 1H, J=5.6); ¹³C NMR (CDCl₃) δ 24.6, 44.4, 45.7, 59.7, 96.6, 98.0, 101.5, 106.4, 114.5, 146.2, 146.6, 148.9, 149.9, 150.5.; HRMS calcd for C₁₅H₁₉N₃O₂: 273.1477; found 273.1473.

### Example 12.b

2-[[[N-(6,7-Methylenedioxyquinolin-4-yl)]amino]methyl]tetrahydrofuran was prepared from tetrahydofurfurylamine (1.01 g, 10 0 mmol) in 84% yield with a reaction time of 20 h. Compound 6g had: mp 276-278° C.; ¹H NMR (CD₃OD) δ 1.77 (m, 1H), 2.07 (m, 3H), 3.61 (m, 2H), 3.86 (m, 2H), 4.26 (m, 1H), 6.28 (s, 2H), 6.90 (d, 1H, J=7.1), 7.19 (s, 1H), 7.74 (s, 1H), 8.21 (d, 1H, J=7.1); ¹³C NMR (CDCl₃) δ 24.7, 28.1, 46.6, 67.3, 76.7, 96.5, 97.6, 97.8, 103.1, 112.2, 135.8, 138.6, 148.3, 153.2, 155.1; HRMS calcd for C₁₅C₁₆N₂O₃: 272.1161; found 272.1172.

The intermediate 4-Chloro-6,7-methylenedioxyquinoline was prepared as follows.

Diethyl 3,4-methylenedioxyanilinomethylene malonate. 3,4-Methylenedioxyaniline (41.0 g, 0 3 mmol) and diethyl ethoxymethylenemalonate (64.8g, 0.3 mmol) were refluxed in benzene for 3.5 hours. The solvent was evaporated in vacuo and the residue was washed with petroleum ether to give 88.3 g as a shiny grey- brown solid, in 96% yield; mp 99.5-101.0° C. (lit.²²¹ mp 102° C.); ¹H NMR (CDCl₃) δ 1.34 (t, 3H, J=7.0), 1.40 (t, 3H, J=7.0) 4.25 (q, 2H, J=7.0), 4.31 (q, 2H, J=7.0), 6.01 (s, 2H), 6.60 (dd, 1H, J=8.5, J=2.2), 6.71 (d, 1H, J=2.2), 6.81 (d, 1H, J=8.5), 8.41 (d, 1H, J=14.0); ¹³C NMR (CDCl₃) δ 14.4, 14.6, 60.1, 60.4, 92.9, 99.4, 101.8, 108.9, 110.9, 134.3, 145.3, 148.9, 152.6, 165.8, 169.3.

4-Hydroxy-6,7-methylenedioxy-3-quinolinecarboxylic acid ethyl ester. Diethyl 3,4-methylenedioxyanilinomethylene malonate (80.0 g, 0.261 mol) was stirred in polyphosphate ester (PPE) (250 g, 0.528 mol) at 120° C. with a mechanical stirrer for 2 hours. The reaction mixture was poured into ice water (700mL) and stirred until homogenous. The mixture was then neutralized (pH 8) with ammonium hydroxide, and the precipitate was filtered, washed well with water, and dried to give 54.7 g as a brown solid, in 80% yield; mp 277-278° C.; ¹H NMR (DMSO-d₆) δ 1.26 (t, 3H, J=7.0), 4.16 (q, 2H, J=7.0), 6.09 (s, 2H), 7.02 (s, 1H), 7.38 (s, 1H), 8.48 (s, 1H).

4-Hydroxy-6,7-methylenedioxy-3-quinolinecarboxylic acid. 4-Hydroxy-6,7-methylenedioxy-3-quinolinecarboxylic acid ethyl ester (45.0 g, 0.172 mol) was added to a solution of KOH (16.8 g, 0.258 mol) in ethanol (500 mL) and the mixture was heated to reflux with stirring for 20 hours. The reaction flask was then cooled and ethanol was evaporated under reduced pressure. Then 800 mL of water were added with stirring to fully dissolve the potassium salt, and the solution was filtered to remove any impurities. Concentrated HCl was added to bring the mixture to pH 1, and the free acid was filtered off and dried under vacuum, to give 33.9 g as a beige solid, in 84%; mp>300° C. (lit.²²¹ mp>290° C); ¹H NMR (DMSO-d₆) δ 6.27 (s, 2H), 7.30 (s, 1H), 7.55 (s, 1H), 8.72 (s, 1H); ¹³C NMR (DMSO-d₆) δ 98.5, 101.8, 103.8, 107.9, 120.8, 137.9, 143.5, 148.1, 153.7, 167.4, 177.4.

6,7-Methylenedioxy-4-quinolone. A suspension of 4-hydroxy-6,7-methylenedioxy-3-quinolinecarboxylic acid (30 g, 0.129 mol) in diphenyl ether (320 mL) was heated to reflux with vigorous stirring. The reaction was carefully monitored until it became clear, about 1.5 h, and then immediately removed from heat. By this time all of the starting material had dissolved but a black tarry residue remained. The solution was decanted and cooled, allowing the product to precipitate. This material was filtered and washed with ethyl ether to remove all traces of phenyl ether. A second crop was obtained by vigorously washing the tarry residue with ethanol (16×250 mL), filtering and evaporating the ethanol, and rinsing the material with ethyl ether. The total yield was 14.9 g as a pale yellow solid, in 61%; mp 285-289° C. (lit.²²¹ mp 276° C.); ¹H NMR (DMSO-d₆) δ 5.95 (d, 1H, J=7.3), 6.13 (s, 2H), 6.97 (s, 1H), 7.38 (s, 1H), 7.77 (d, 1H, J=7.3); ¹³C NMR (DMSO-d₆) δ 97.5, 102.1, 102.6, 108.7, 119.4, 122.0, 130.8, 138.7, 145.8, 151.7.

4-Chloro-6,7-methylenedioxyquinoline. 6,7-Methylenedioxy-4-quinolone (5.0 g, 26.5 mmol) was boiled in POCl₃ (75 mL) for 45 min and then cooled. Excess phospohoryl chloride was removed under reduced pressure and ice water (100 mL) was added to hydrolyze any residual phosphoryl chloride. The mixture was basified (pH 9) with ammonium hydroxide, and the solid precipitate was filtered. This material was extracted into ethyl ether (8×100 mL), and the ether solution was dried (MgSO₄) and evaporated to provide 4.55 g as a white solid, in 83%; mp 127.5-128° C. (lit. mp 129° C.); ¹H NMR (CDCl₃) δ 6.15 (s, 2H), 7.35 (d, 1H, J=4.7), 7.39 (s, 1H), 7.49 (s, 1H), 8.56 (d, 1H, J=4.7); ¹³C NMR (CDCl₃) δ 99.8, 102.2, 106.1, 119.9, 123.7, 129.8, 141.2, 147.7, 149.1, 151.4.

### Examples 13-16

The representative compounds of the invention at Examples 13-16 were prepared by deprotection of the corresponding tert-butyldimethylsilyl ethers (13-15) or the corresponding acetal as described below.

### Example 13

8,9-Dimethoxy-2,3-methylenedioxy-5-[2-(hydroxy)ethyl]-5H-dibenzo[c,h]1,6-naphthyridin-6-one: Prepared from the corresponding tert-butyldimethylsilyl ether (Example 13.a.) by treatment with AcOH, THF, H₂O (3:1:1) at room temperature; (84% yield); reaction time 48 h; mp 285-286° C.; IR (KBr); 1653, 3448; ¹H NMR (DMSO-d₆); δ 3.91 (s, 3H), 4.04 (s, 3H), 4.54 (t, 2H, J=4.4), 4.96 (t, 2H, J=4), 6.26 (s, 2H), 7.44 (s, 1H), 7.71 (s, 1H), 7.98 (s, 1H), 8.03 (s, 1H), 9.64 (s, 1H); ¹³C NMR (DMSO-d₆); δ 52.6, 56.4, 57.0, 59.5, 101.9, 103.0, 104.0, 106.8, 108.8, 111.9, 114.8, 119.1, 128.0, 141.2, 144.9, 147.4, 147.7, 150.2, 150.5, 154.6, 163.7; HRMS calcd (M⁺—OH)for C₂₁H₁₇O₅N₂ 377.1137; Found 377.1121.

### Example 14

8,9-Dimethoxy-2,3-methylenedioxy-5-[2-(2-hydroxyethoxy)ethyl]-5H-dibenzo[c,h]1,6-naphthyridin-6-one: Prepared from the corresponding tert-butyldimethylsilyl ether (Example 14.a.) by treatment by treatment with AcOH, THF, H₂O (3:1:1) at room temperature; (76% yield); reaction time 18 h; mp 235° C.; IR (KBr) 1654; ¹H NMR (CDCl₃); δ 3.61 (t, 2H, J=5.2), 3.73 (t, 2H, J=5.2), 4.07 (s, 3H), 4.14 (s,3H), 4.22 (t, 2H, J=5.6), 4.71 (t, 2H, J=5.6), 6.2 (s, 2H), 7.53 (s, 1H), 7.69 (s, 1H), 7.88 (s, 1H), 8.05 (s, 1H), 9.39 (s, 1H). HRMS calcd for C₂₃H₂₂N₂O₇H: 439.1506; found 439.1499.

### Example 15

8,9-Dimethoxy-2,3-methylenedioxy-5-[2-N,N-dimethylamino-1-(hydroxymethyl)ethyl]-5H-dibenzo[c,h]1,6-naphthyridin-6-one: Prepared from the corresponding tert-butyldimethylsilyl ether (Example 15.a.) by treatment with 5N HCl in isopropanol at room temperature for 30 min; (57% yield); reaction time 30 min; mp 132° C.; IR (KBr) 1647; ¹H NMR (CDCl₃); 8 2.00 (s, 6H), 2.72-2.81 (m, 1H), 3.16-3.26 (m, 1H), 4.05 (s, 3H), 4.12 (s, 3H), 4.20-4.28 (m, 1H), 4.65-4.73 (m, 1H), 4.98 (m, 1H), 6.17 (q, 2H, J=1.2), 7.44 (s, 1H), 7.51 (s, 1H), 7.64 (s, 1H), 7.82 (s, 1H), 7.82 (s, 1H); 9.33 (s, 1H); ¹³C NMR (CDCl₃) δ: 45.6, 56.2, 56.3, 60.0, 64.1, 65.2, 100.9, 101.8, 102.3, 106.6, 108.5, 112.5, 115.0, 119.6, 127.5, 141.1, 143.0, 147.1, 147.5, 149.9, 150.0, 154.1, 165.0.

### Example 16

8,9-Dimethoxy-2,3-methylenedioxy-5-[2,3-dihydroxy)propyl]-5H-dibenzo[c,h]1,6-naphthyridin-6-one: Prepared from the corresponding acetal (Example 16.a.) by treatment 80% AcOH at reflux for 2 h. The reaction mixture was allowed to cool, and then concentrated in vacuo. The crude residue was triturated with chloroform (1.5 mL), filtered, and washed with additional chloroform (10 mL), to provide 16.5 mg of pure material, in 60% yield; mp 272-274° C. (dec.); IR (KBr) 1631, 3407; ¹H NMR (DMSO-d₆) δ 3.31 (d, 2H, J=8.0), 3.95 (s, 3H), 4.07 (s, 3H), 4.63 (m, 3H), 6.33 (s, 2H), 7.55 (s, 1H), 7.72 (s, 1H), 8.06 (s, 2H), 8.21 (s, 1H), 9.79 (s, 1H); ¹³C NMR (DMSO-d₆) δ 54.4, 56.5, 57.3, 64.9, 68.8, 103.2, 103.8, 104.6, 108.9, 109.0, 112.6, 115.5, 119.3, 127.3, 138.5, 140.6, 148.2, 151.0, 151.3, 151.8, 154.8, 163.9; HRMS calcd for C₂₂H₂₀N₂O₇H: 425.1350; found 425.1359.

### Examples 13.a-16.a

The intermediate iodo compounds of Examples 13.b.-16.b. were cyclized using the following general procedure.

A mixture of the requsite 4-amino-6,7-methylenedioxyquinoline o-iodobenzamide derivative (1.0 mmol equiv.), Pd(OAc)₂ (0.2 mmol equiv.), P(o-tolyl)₃ (0.4 mmol equiv.), and Ag₂CO₃ (2.0 mmol equiv) was heated to reflux in DMF (30 mL per mmol equiv.) with stirring. The reaction mixture was allowed to cool to room temperature, diluted with CHCl₃, and filtered through Celite. The sicciate was extensively washed with 10% CH₃OH in CHCl₃. The filtrate was concentrated in vacuo and the residue chromatographed on silica gel using chloroform:methanol.

### Example 13.a

Prepared from N-(6,7-Methylenedioxyquinolin-4-yl)-N-[(2-(t-butyldimethylsilanyloxy)-ethyl]-2-iodo-4,5-dimethoxybenzamide (36.4% yield); reaction time 30 min; mp 271-273° C.; IR (KBr) 1658; ¹H NMR (CDCl₃) δ 0.00 (s, 6H), 0.68 (s, 9H), 4.04 (s, 3H), 4.12 (s, 3H), 4.24 (t, 2H, J=8), 4.65 (t, 2H, J=8), 6.18 (s, 2H), 7.44 (s, 1H), 7.64 (s, 1H), 7.85 (s, 1H), 8.01 (s, 1H), 9.29 (s, 1H); HRMS calcd for C₂₇H₃₃ISiN₂O₆H: 637.1153; found 637.1212

### Example 14.a

Prepared from N-(6,7-Methylenedioxyquinolin-4-yl)-N-[2-(2-(t-butyldimethylsilanyloxy)ethoxy)ethyl]-2-iodo-4,5-dimethoxybenzamide; (75% yield); reaction time 18 h; mp 238° C. (dec.); IR (KBr): 1639; ¹H NMR (CDCl₃); δ 0.00 (s, 6H), 0.85 (s, 9H), 3.54 (t, 2H, J=5.2), 3.70 (t, 2H, J=5.2), 4.07 (s, 3H), 4.14 (s,3H), 4.16 (t, 2H, J=6.0), 4.71 (t, 2H, J=6.0), 6.17 (s, 2H), 7.48 (s, 1H) 7.70 (s, 1H), 7.94 (s, 1H), 9.39 (s, 1H); HRMS calcd for C₂₃H₂₃N₂O₇H: 439.1505; found 439.1506.

### Example 15.a

Prepared from N-(6,7-Methylenedioxyquinolin-4-yl)-N-[1-[(t-butyldimethylsilanyloxy)-methyl]-N-2-dimethylaminoethyl]]-2-iodo-4,5-dimethoxybenzamide (95% yield); reaction time 45 min; ¹H NMR (CDCl₃); δ−0.13 (6H), 069 (s, 9H), 1.97(s, 6H), 1.92 (s, 6H), 2.52 (m, 1H), 2.80 (m, 1H) 3.20 (m, 1H), 4.01 (s, 3H), 4.09(s, 3H), 4.50 (m, 1H), 4.90 (m, 1H), 6.11 (m,2H), 7.30 (s, 1H), 7.61 (s, 1H) , 7.79 (s, 1H), 8.19 (s, 1H), 9.32 (s, 1H).

### Example 16.a

8,9-Dimethoxy-2,3-methylenedioxy-5-[2,2-dimethyl[1,3]dioxolan-4-yl]methyl]-5H-dibenzo[c,h]1,6-naphthyridin-6-one was prepared from N-(6,7-Methylenedioxyquinolin-4-yl)-N-[(2,3-dihydroxy)propyl]-2-iodo-5,6-dimethoxybenzamide (22% yield); reaction time 45 min); mp 241-244° C. (dec.); IR (CHCl₃) 1652; ¹H NMR (CDCl₃) δ 1.34 (s, 3H), 1.36 (s, 3H), 3.95 (m, 2H), 4.08 (s, 3H), 4.14 (s, 3H), 4.35 (m, 1H), 4.55 (m, 1H), 4.77 (m, 1H), 6.19 (s, 2H), 7.48 (s, 1H), 7.70 (s, 1H), 7.87 (s, 2H), 8.05 (s, 1H), 9.40 (s, 1H); ¹³C NMR (CDCl₃) δ 25.5, 26.5, 54.0, 56.3, 56.4, 69.4, 75.5, 101.6, 102.1, 102.3, 107.0, 108.7, 109.7, 111.8, 114.9, 119.1, 127.8, 141.1, 143.5, 147.4, 147.7, 150.1, 150.4, 154.4, 164.6; HRMS calcd for C₂₅H₂₄N₂O₇H 465.1662; found 435.1677. The compound 8,9-Dimethoxy-2,3-methylenedioxy-5-[2,2-dimethyl[1,3]dioxolan-4-yl]methyl]-5H-dibenzo[c,h]1,6-naphthyridin-6-one is also a compound of the invention.

### Examples 13.b.-16.b

The intermediate 4-amino-6,7-methylenedioxyquinoline o-iodobenzamide derivatives used in Examples 13.a.-16.a. were prepared using the following general procedure.

A 2.0M solution of oxalyl chloride in CH₂Cl₂(1.3 equiv.) was added to a solution of 2-iodo-5,6-dimethoxybenzoic acid (1.0 equiv.) in anhydrous CH₂Cl₂ (≈60 mL per 10 mmol benzoic acid) and the solution stirred at reflux for 3 h. The mixture was allowed to cool and was then concentrated to dryness in vacuo. To the residue was added a solution of appropriate 4-amino-6,7-dimethoxyquinoline (1.0 equiv), triethylamine (2 equiv.) in CH₂Cl₂(≈60 mL per 4 mmol aminoquinoline). The reaction mixture was then stirred at reflux under N₂. In the case of those derivatives that have an alkylamine incorporated in their structure, the residue was partitioned between CHCl₃ and 10% NaOH. The aqueous layer was repeatedly separated with CHCl₃. All of the CHCl₃ solutions (initial partition and extracts) were combined and dried (MgSO₄). The aqueous layer was neutralized with 20% NaOH and extracted with CHCl₃, dried (MgSO₄) and evaporated.

### Example 13.b

N-(6,7-Methylenedioxyquinolin-4-yl)-N-[(2-(t-butyldimethylsilanyloxy)-ethyl]-2-iodo-4,5-dimethoxybenzamide. Prepared from 4-[N-[2-(t-Butyldimethylsilanyloxy)]ethyllamino-6,7-methylenedioxyquinoline (400 mg, 1.15 mmol) in 51.7% yield with a reaction time of 12 h, from the acid chloride prepared using 5.0 mmol of oxalyl chloride and 1.38 mmol of 2-iodo-5,6-dimethoxybenzoic acid. Compound 8h had: mp 79-80° C.; IR (KBr); 1653 ¹H NMR (CDCl₃); δ 0.004 (d, 3H, J=4.2Hz), 0.82 (s, 9H), 3.26 (s, 3H), 3.67 (s, 3H), 3.84-4.02 (m, 4H), 6.13 (d, 2H, J=4 Hz), 6.40 (s, 1H), 7.02 (s, 1H), 7.33 (d, 1H, J=4.2Hz), 7.36 (s, 1H), 7.42 (s, 1H), 8.52 (d, 1H, J=4Hz); HRMS calcd for C₂₇H₃₃ISiN₂O₆H 637.1232; observed 637.1212

### Example 14.b

N-(6,7-Methylenedioxyquinolin-4-yl)-N-[2-(2-(t-butyldimethylsilanyloxy)ethoxy)ethyl]-2-iodo-4,5-dimethoxybenzamide. Prepared from 4-[N-[2-[2-(t-Butyldimethylsilanyloxy)ethoxy]ethyl]ethyl]amino-6,7-methylenedioxyquinoline (354 mg, 9.0 mmol) in 60% yield with a reaction time of 24 h, from the acid chloride prepared using 4.5 mmol of oxalyl chloride and 1.8 mmol of 2-iodo-5,6-dimethoxybenzoic acid. Compound 8i had: ¹H NMR (CDCl₃); δ 0.006 (s, 6H), 0.83 (s, 9H), 3.27 (s, 3H), 3.48 (t, 2H, J=4.6), 3.67 (t, 2H, J=5.6), 3.69 (s, 3H), 3.76-4.55 (m, 4H), 6.10 (s, 2H), 6.36 (s, 1H), 6.99 (s, 1H), 7.30-7.32 (three singlets, 3H), 8.52 (d, 1H, J=4.8).

### Example 15.b

N-(6,7-Methylenedioxyquinolin-4-yl)-N-[1-[(t-butyldimethylsilanyloxy)-methyl]-N-2-dimethylaminoethyl]]-2-iodo-4,5-dimethoxybenzamide. Prepared from 4-[N-4-[2-(N,N-dimethylamino)-1-[(t-butyldimethylsilanyloxy)methyl]-ethyl]amino-6,7-methylenedioxyquinoline (0.48 mg, 1.2 mol) in 55% yield with a reaction time of 18 h, from the acid chloride prepared using 5.9 mmol of oxalyl chloride and 2.4 mmol of 2-iodo-5,6-dimethoxybenzoic acid. Compound 8j had: IR (CHCl₃) 1656; ¹H NMR (CDCl₃) [unresolved atropisomers in a an apparent 57:43 ratio ar r.t.] major atropisomer δ 0.01 (s, 6H), 0.84 (s, 9H), 2.34 (s, 6H), 2.55 (m, 1H), 2.85 (m, 1H); 3.43 (s, 3H), 3.71(s, 3H) 3.86-4.04 (m, 3H), 6.12 (s, 2H), 6.56 (s, 1H), 7.29-7.31 (s, 1H), 7.67 (d, 1H, J=5.0), 8.00 (s, 1H), 8.59 (d, 1H, J=4.4); minor atropisomer 6 0.17 (s, 6H), 0.96 (s, 9H), 2.15 (s, 6H), 2.55 (m, 1H), 2.85 (m, 1H), 3.36 (s, 3H), 3.72 (s, 3H) 3.86-4.04 (m, 3H), 6.13 (s, 2H), 6.53(s, 1H), 7.00 (s, 1H), 7.31 (s, 1H), 7.51 (d, 1H, J=4.8), 8.25 (s, 1H), 8.55 (d, 1H, J=5.2).

### Example 16.b

N-(6,7-Methylenedioxyquinolin-4-yl)-N-[(2,3-dihydroxy)propyl]-2-iodo-5,6-dimethoxybenzamide. Prepared from 4-[N-(2,2-dimethyl-[1,3]dioxolan-4-yl)methyl]amino-6,7-methylenedioxyquinoline (290 mg, 0 9 mmol) in 47% yield with a reaction time of 12 h, from the acid chloride prepared using 30 mmol of oxalyl chloride and 13 mmol of 2-iodo-5,6-dimethoxybenzoic acid. The acid chloride was added as a methylene chloride solution to a solution of 7k in 125 mL of DME containing triethylamine (3.04 g 30.1 mmol). Compound 8k had: IR (CHCl₃) 1653; ¹H NMR (CDCl₃) δ 1.21 (s, 3H), 1.33 (s, 3H), 3.33 (s, 3H), 3.76 (s, 3H), 3.94 (m, 3H), 4.61 (m, 2H), 6.18 (s, 1H), 6.39 (s, 1H), 7.05 (s, 1H), 7.31 (d, 1H, J=4.8), 7.46 (s, 1H), 7.49 (s, 1H), 8.61 (d, 1H, J=4.8); ¹³C NMR (CDCl₃) δ 25.6, 26.9, 55.6, 56.1, 56.4, 68.2, 73.2, 82.8, 98.2, 98.7, 102.4, 106.1, 110.3, 120.7, 121.7, 124.1, 133.3, 147.5, 148.0, 148.8, 149.5, 150.0, 151.5, 152.3, 167.8; HRMS calcd for C₂₅H₂₅N₂O₇IH: 593.0785; found 593.0802.

### Examples 13.c.-15.c

The intermediate alcohols from Examples 13.d.-15.d. were converted to their corresponding silyl ethers using the following general procedure.

A mixture of the 4-amino-6,7-methylenedioxyquinoline derivative (1.0 mmole equiv.), imidazole (1.1 mmol equiv.) and t-butyldimethylsilyl chloride (1.2 mmol equiv.) in DMF (15 mL per mmol equiv) was stirred at room temperature for 6 h. DMF was removed in vacuo, water was added to residue, and solid was filtered and dried.

### Example 13.c

4-[N-[2-(t-Butyldimethylsilanyloxy)]ethyl]amino-6,7-methylenedioxyquinoline. Prepared from N-(6,7-Methylenedioxyquinolin-4-yl)ethanolamine in 48.7% yield; mp 215-216° C.; ¹H NMR (DMSO-d₆) δ 0.01 (s, 6H), 0.85 (s, 9H), 3.39 (dd, 2H, J=6, 12), 3.80 (t, 2H, J=6.2), 6.14 (s, 2H), 6.42 (d, 1H, J=5.4), 7.12 (s, 1H), 7.60 (s, 1H), 8.18 (d, 1H, J=4.8).

### Example 14.c

4-[N-[2-[2-(t-Butyldimethylsilanyloxy)ethoxy]ethyl]ethyl]amino-6,7-methylenedioxyquinoline. Prepared from 2-[2-[N-(6,7-Methylenedioxyquinolin-4-yl)]amino]ethoxyethanol in 39% yield (overall yield from 5); ¹H NMR (CDCl₃) δ 0.1 (s, 6H), 0.92 (s, 9H), 3.64-3.69 (m, 4H), 3.84 (d, 2H, J=5.2,), 3.93 (d, 2H, J=5.2), 6.15 (s, 2H), 6.56 (d, 1H, J=6.4), 7.42 (s, 1H), 7.82 (s, 1H), 8.18 (d, 1H, J=6.4).

### Example 15.c

4-[N-4-[2-(N,N-dimethylamino)-1-[(t-butyldimethylsilanyloxy)methyl]-ethyl]amino-6,7-methylenedioxyquinoline. Prepared from 2-[[N-(6,7-Methylenedioxyquinolin-4-yl)]amino]-3-(N,N-dimethylamino)propanol in 25% yield (overall yield from 5); ¹H NMR (CDCl₃) [unresolved atropisomers in a an apparent 57:43 ratio at r.t.] major atropisomer δ 0.07(s, 6H), 0.92-0.94 (s, 9H), 2.24 (s, 6H), 2.45-2.55 (m, 2H), 3.60-4.05 (m, 3H), 5.40 (d, 1H), 6.09 (s, 2H), 6.45 (d, 1H, J=6.4), 7.02 (s, 1H), 7.30 (s, 1H), 8.18 (d, 1H, J=6.4); minor atropisomer 8 0.09 (s, 6H), 0.94 (s, 9H), 2.30 (s, 6H), 2.45-2.55 (m, 2H), 3.60-4.05 (m, 3H), 5.40 (d, 1H), 6.0 (s, 2H), 6.45 (d, 1H, J=6.4), 7.02 (s, 1H), 7.30 (s, 1H), 8.18 (d, 1H, J=6.4)

### Example 16.c

4-[N-(2,2-dimethyl-[1,3]dioxolan-4-yl)methyl]amino-6,7-methylenedioxyquinoline. A mixture of 34[N-(6,7-Methylenedioxyquinolin-4-yl)]amino]-1,2-propandiol (500 mg, 1.9 mmol), p-toluenesulfonic acid (5 mg, 0.02 mg) in DMF (20 mL) and 2,2-dimethoxypropane (5 mL), was heated to 80° C. and stirred at this temperature for 18 h. To the cooled solution was added 1 mL of pyridine and the solvent evaporated in vacuo. The crude material was chromatographed in 96:4 chloroform-methanol to give 466 mg of the acetonide, in 81% yield; mp 219-221° C.; ¹H NMR (CD₃OD) δ 1.35 (s, 3H), 1.38 (s, 3H), 3.74 (m, 3H), 4.19 (m, 1H), 4.49 (m, 1H), 6.28 (s, 2H), 6.94 (d, 1H, J=7.2), 7.20 (s, 1H), 7.74 (s, 1H), 8.24 (d, 1H, J=7.2); ¹³C NMR (CD₃OD) δ 23.5, 25.1, 45.0, 66.0, 73.6, 96.5, 97.7, 97.8, 103.1, 109.1, 112.2, 135.8, 138.6, 148.4, 153.3, 155.3; HRMS calcd for C₁₆H₁₈N₂O₄: 302.1267; found 302.1267.

### Examples 13.d-16.d

The intermediate 4-amino-6,7-dimethoxyquinoline derivatives used in Examples 13.c-16.c. were prepared using the following general procedure.

4-Chloro-6,7-methylenedioxyquinoline was stirred in refluxing phenol (5.5 mol equiv.) for 2.5 h. The temperature was lowered to 100° C. and the primary amine (1.0 mol equiv.) added with stirring. The reaction was then allowed to stir at 100° C. for several hours, and the phenol removed by Kugelrohr distillation under reduced pressure. In the case of those derivatives that have an alkylamine incorporated in their structure, the residue was partitioned between CHCl₃ and 10% NaOH. The aqueous layer was repeatedly separated with CHCl₃. All of the CHCl₃ solutions (initial partition and extracts) were combined and dried (MgSO₄). Other 4-amino-6,7-methylenedioxyquinoline derivatives were purified by column chromatography.

### Example 13.d

N-(6,7-Methylenedioxyquinolin-4-yDethanolamine was prepared from ethanolamine (0.6 g, 10 mmol) from in 53.9% yield with a reaction time of 24 h: mp 233-234° C.; ¹HNMR (DMSO-d₆); δ 3.51 (dd, 2H, J=10.4, 6.), 3.69 (t, 2H, J=6.0), 6.27 (s, 2H), 6.72 (d, 1H, J=7.0), 7.37 (s, 1H), 8.12 (s, 1H), 8.29 (d, 1H, J=7.0); ¹³C NMR (DMS)-d₆); 46.5, 59.5, 98.6, 98.8, 100.3, 103.8, 113.2, 137.6, 141.0, 148.2, 152.8, 155.0; HRMS calcd for C₁₂H₁₂N₂O₃H: 232.0848; found 232.0881.

### Example 14.d

2-[2-[N-(6,7-Methylenedioxyquinolin-4-yl)]amino]ethoxyethanol was prepared from 2-[2-(hydroxyethyl)ethoxy]ethylamine (0.76 g, 7 2 mmol) with a reaction time of 18 h. The compound was converted directly to its t-butyldimethylsilanyloxy derivative in Example 14.c. above.

### Example 15.d

2-[[N-(6,7-Methylenedioxyquinolin-4-yl)]amino]-3-(N,N-dimethylamino)propanol was prepared from 1-(hydroxymethyl)-2-(N,N-dimethylethylenediamine (1.13 g, 9.6 mmol) with a reaction time of 48 h. The compound was converted directly to its t-butyldimethylsilanyloxy derivative in Example 15.c. above.

### Example 16.d

3-[[N-(6,7-Methylenedioxyquinolin-4-yl)]amino]-1,2-propandiol was prepared from 3-amino-1,2-propanediol (1.32 g, 14.5 mmol) in 34% yield with a reaction time of 24 h: mp 213-217° C. (dec.); ¹H NMR (CD₃OD) δ 3.67 (m, 5H), 6.26 (s, 2H), 6.87 (d, 1H, J=7.2), 7.19 (s, 1H), 7.71 (s, 1H), 8.21 (d, 1H, J=7.2); ¹³C NMR (CD₃OD) δ 45.7, 63.1, 69.4, 96.8, 97.4, 97.8, 103.0, 112.3, 136.1, 138.9, 148.2, 153.0, 155.0; HRMS calcd for C₉H₇N₃O₂: 262.0954; found 262.0954.

### Example 17

### 8,9-Dimethoxy-2,3-methylenedioxy-5-[2-(N,N-dimethylamino)ethyl]-5,6-dihydro-dibenzo[c,h]1,6-naphthyridine (4a)

To a solution of 8,9-dimethoxy-2,3-methylenedioxy-5-[2-(N,N-dimethylamino)ethyl]-5H-dibenzo[c,h]1,6-naphthyridin-6-one (160 mg, 0.38 mmol) in THF (650 mL) was added LiA1H₄ (75 mg, 2.0 mmol), and the mixture was stirred under nitrogen at reflux. After 2 h, an additional 20 mmol of LiAlH₄ was again added. The reaction was refluxed for an additional 3 h, then allowed to cool to room temperature. The reaction was quenched by the sequential addition of water (5 drops), 10% NaOH (5 drops), and water (5 drops). The mixture was filtered through Celite and evaporated, and the crude mixture was chromatographed on silica in 98:2 chloroform-methanol, to give 132 mg of the reduced product, in 85% yield; mp 271-273° C. (dec.); ¹H NMR (CDCl₃) δ 2.24 (s, 6H), 2.58 (t, 2H, J=6.8), 3.12 (t, 2H, J=6.8), 3.97 (s, 3H), 4.02 (s, 3H), 4.27 (s, 2H), 6.13 (s, 2H), 6.79 (s, 1H), 7.38 (s, 2H), 7.61 (s, 1H), 9.05 (s, 1H); ¹³NMR (CDCl₃) δ 46.0, 50.6, 51.2, 56.2, 26.3, 58.4, 99.6, 101.7, 105.7, 106.6, 110.0, 120.7, 123.1, 124.8, 131.1, 144.1, 146.9, 148.0, 149.0, 149.4, 149.8, 150.2; HRMS calcd for C₂₃H₂₅N₃O₄: 407.1845; found 407.1848.

### Example 18

8,9-Dimethoxy-2,3-methylenedioxy-5-[2-(N,N-dimethylamino)-1-methylethyl]-5,6-dihydro-dibenzo[c,h]1,6-naphthyridine. The title compound was prepared as follows. 8,9-Dimethoxy-2,3-methylenedioxy-5-[2-(N,N-dimethylamino)-1-methylethyl]-5H-dibenzo[c,h]1,6-naphthyridin-6-one (80 mg, 0.18 mmol; Example 7) in THF (150 mL) was added to LiAlH₄ (50 mg, 1 3 mmol), and the mixture was stirred under nitrogen at reflux for 4 h. The reaction was quenched by the sequential addition of water (5 drops), 10% NaOH (5 drops), and water (5 drops). The mixture was filtered through Celite and evaporated, and the crude mixture was chromatographed on silica in 1.0% methanol in chloroform to give 35 mg of the reduced product, in 45.4% yield; mp 153-154° C.; ¹H NMR (CDCl₃) δ 1.16 (d, 3H, J=8), 2.38 (dd, 2H, J=12.2, 8.0), 3.68-3.80 (m, 1), 3.88 (s, 3H), 4.24 (s, 2H), 6.16 (s, 2H), 6.64 (s, 1H), 7.24 (s, 1H), 7.40 (s, 214), 7.62 (s, 1H), 8.88 (s, 1H); ¹³C NMR (CDCl₃) δ: 17.7, 45.6, 46.0, 56.2, 56.4, 57.8, 64.2, 100.1, 101.7, 105.8, 106.4, 108.5, 120.5, 120.6, 123.6, 126.9, 143.4, 146.6, 147.7, 148.9, 149.5, 149.6, 150.0; HRMS calcd for C₂₄H₂₇N₃O₄H 422.2002; found 422.2081.

### Example 19

### 8,9-Dimethoxy-2,3-methylenedioxy-5-[2-(N,N-diethylamino)ethyl]-5H-dibenzo[c,h]1,6-naphthyridin-6-one

A mixture of N-(6,7-Methylenedioxyquinolin-4-yl)-N-[2-(N,N-diethylamino)ethyl]-2-iodo-4,5-dimethoxybenzamide (577 mg, 1.0 mmol), Pd(OAc)₂ (45, 0.2 mmol), P(o-tolyl)₃ (122 mg, 0.4 mmol), and silver carbonate (550 mg, 2.0 mmol) was heated to reflux in DMF (30 mL) and stirred under nitrogen for 30 minutes. The reaction mixture was cooled to room temperature, diluted with chloroform and filtered though a bed of Celite. The filter was washed well with 90:10 chloroform-methanol. Then the solvent was removed under reduced pressure and the resulting residue was chromatographed on silica gel using 99:1 chloroform-methanol to give the cyclized compound (250 mg) as a white solid, in 56% yield; mp 221-223° C. (dec.); IR (CHCl₃) 3029, 3009, 2971, 2939, 2910, 1648, 1611, 1570, 1523, 1497, 1467, 1386, 1310, 1267, 1248, 1217, 1213, 1166, 1040; ¹H NMR (CDCl₃) δ 0.95 (t, 6H, J=7.0), 2.80 (1, 4H, J=7.0), 3.04 (t, 2H, J=6.7), 4.06 (s, 3H), 4.13 (s, 3H), 4.63 (t, 2H, J=6.7), 6.17 (s, 2H), 7.46 (s, 1H), 7.68 (s, 1H), 7.90 (s, 1H), 7.96 (s, 1H), 9.37 (s, 1H); ¹³C NMR (CDCl₃) δ 12.0, 47.6, 49.6, 51.7, 56.3, 101.4, 102.0, 102.2, 107.0, 108.9, 111.8, 115.0, 119.5, 127.7, 141.1, 143.5, 147.3, 147.7, 149.9, 150.3, 154.2, 164.2; HRMS calcd for C₂₅H₂₇O₅N₃H: 450.2030; found: 450.2032.

a. 4-[[2-(Diethylamino)ethyl]amino]-6,7-methylenedioxyquinoline. 4-Chloro-6,7-methylenedioxyquinoline. (1.0 g, 4.83 mmol) was stirred in boiling phenol for 2.5 hours. Then the mixture was cooled to 140° C. and N,N-diethylethylenediamine (1.16 g, 10.0 mmol) was added. The reaction mixture was stirred at this temperature for 18 hours, and then phenol was removed on the Kugelrohr. The crude residue was partitioned between dilute HCl (100 mL) and chloroform (100 mL), and the organic phase was extracted with dilute HCl (100 mL). The combined aqueous phases were washed with chloroform (100 mL) and then basified with 30% NaOH, extracted into chloroform (3×100 mL), dried (MgSO₄) and evaporated to give 793 mg as a white solid, in 58% yield; mp 201-202° C.; IR (CHCl₃) 3364, 2967, 2936, 2907, 2875, 1620, 1546, 1466, 1295, 1222, 1218, 1210, 1152, 1041; ¹H NMR (CDCl₃) δ 1.09 (t, 6H, J=7.2), 2.61 (q, 4H, J=7.2), 2.82 (t, 2H, J=5.8), 3.26 (m, 2H), 5.71 (br, 1H), 6.08 (d, 2H), 6.35 (d, 1H, J=5.2), 7.03 (s, 1H), 7.31 (s, 1H), 8.40 (d, 1H, J=5.2); ¹³C NMR (CDCl₃) δ 12.2, 40.1, 46.7, 51.0, 96.1, 99.0, 101.5, 106.7, 114.5, 146.5, 146.7, 149.1, 149.6, 149.9; HRMS calcd for C₁₆H₂₁O₂N₃: 287.1634; found: 287.1631.

b. N-(6,7-Methylenedioxyquinolin-4-yl)-N-[2-(N,N-diethylamino)ethyl]-2-iodo-4,5-dimethoxybenzamide. Oxalyl chloride (1.12 g, 8.8 mmol) was added to a solution of 2-Iodo-4,5-dimethoxybenzoic acid (820 mg, 2 6 mmol; see above) in anhydrous methylene chloride (40 mL) and the stirred mixture was refluxed for 4 hours. The mixture was then concentrated to dryness under reduced pressure. The acid chloride was dissolved in 40 mL of methylene chloride and added to a solution of 4-[[2-(Diethylamino)ethyl]amino]-6,7-methylenedioxyquinoline (640 mg, 2.2 mmol), and triethylamine (2.2g, 22 mmol) in methylene chloride (50 mL) and the resulting mixture was stirred at reflux under nitrogen for 2 hours. The reaction mix was cooled and washed with a saturated solution of sodium bicarbonate (3×75 mL), and extracted into dilute HCl (4×100 mL). The aqueous extract was then neutralized with 30% NaOH and extracted with CHCl₃ (4×100 mL), washed with brine (100mL), dried (MgSO₄) and evaporated, yielding 1.1 g as a sticky semisolid glue, in 86% yield; ¹H NMR (CDCl₃) δ 0.96 (t, 6H, J=7.2), 2.54 (q, 4H, J=7.2), 2.82 (m, 2H), 3.29 (s, 3H), 3.71 (s, 3H), 3.92 (m, 1H), 4.46 (m, 1H), 6.12 (s, 2H), 6.37 (s, 1H), 7.00 (s, 1H), 7.27 (d, 1H, J=4.8), 7.33 (s, 1H), 7.39 (s, 1H), 8.52 (d, 1H, J=4.8); ¹³C NMR (CDCl₃) 6 11.8, 47.1, 47.5, 50.7, 55.5, 56.1, 82.7, 98.5, 102.2, 106.7, 110.6, 120.1, 121.8, 122.7, 133.7, 146.3, 148.1, 148.3, 148.5, 149.0, 149.7, 151.0, 170.0; HRMS calcd for C₂₅H₂₈O₅N₃IH: 578.1153; found: 578.1153.

The intermediate 4-Chloro-6,7-methylenedioxyquinoline was prepared as described above.

The intermediate 2-Iodo-4,5-dimethoxybenzoic acid was prepared as follows.

c. 2-Iodo-4,5-dimethoxybenzoic acid. A mixture of 2-amino-4,5-dimethoxybenzoic acid (10.0 g, 50 mmol) in water (100 mL) and concentrated H₂SO₄ (14 mL) was cooled to 5° C. and a solution of NaNO₂ (3.5 g) in water (12.5 mL) was added in a dropwise fashion while maintaining the temperature between 0-5° C. Follwing the addition the mixture was stirred at this temperature for an additional 30 minutes. Then a solution of KI (13.0 g, 78.3 mmol) in water (20.5 mL) and concentrated H₂SO₄ (4.4 mL) was rapidly added and the flask was transferred to an oil bath that had been preheated to 105° C. The mixture was stirred for 30 minutes following the onset of reflux. The flask was then cooled and extracted into chloroform (3×300 mL), washed with water (3×200 mL), dilute HCl (200 mL), and brine (200 mL), then the solvent was dried (Na₂SO₄) and evaporated, and the residue was chromatographed in chloroform to give 13.1 g as a white solid, in 84% yield; mp 162.0-163.5° C. (lit. mp 159-160° C.); ¹H NMR (CDCl₃) δ 3.93 (s, 3H), 3.95 (s, 3H), 7.46 (s, 1H), 7.65 (s, 1H); ¹³C NMR (CDCl₃) δ56.1, 56.4, 85.8, 114.8, 124.3, 124.5, 148.8, 152.7, 170.5.

### Example 20

Using procedures similar to those described above, the compound 2,3-dimethoxy-8,9-methylenedioxy-11-[2-(4-methylpiperazin-1-yl)ethyl]-11H-5,6,11-triazachrysen-12-one was also prepared.

### Example 21

Using procedures similar to those described above, the following compounds of the invention were also prepared: 8,9-dimethoxy-2,3-methylenedioxy-5-(2-piperidinoethyl)-5H-dibenzo[c]1,6-naphthyridin-6-one; 8,9-dimethoxy-2,3-methylenedioxy-5-[2-(4-benzylpiperazin-1-yl)ethyl]-5H-dibenzo[c,h]1,6-naphthyridin-6-one; 8,9-dimethoxy-2,3-methylenedioxy-5-formylmethyl-5H-dibenzo[c,M1,6-naphthyridin-6-one; and 8,9-dimethoxy-2,3-methylenedioxy-5-[2-(N-methylamino)ethyl]-5H-dibenzo[c,h]1,6-naphthyridin-6-one.

### Example 22

The in vitro and in vivo activity of compound 2 and two of its metabolites (compound 5 and compound 6) were explored and compared with the activity of camptothecin Top1 inhibitors. In vitro in mouse, rat, dog, and human, compound 2 exhibited high metabolic stability, plasma binding of 88-93% and exhibited concentration dependent partitioning into red blood cells. In vivo, compound 2 had a large volume of distribution and low-to-moderate clearance in mouse, rat and dog. In nude mice, the t_{1/2} for compound 2 was 3.6 h (po), 10.4 h (ip) and 5.1 h (iv) and longer in tumor-bearing mice. In human HCT-116 colon ca, HT-29 colon ca and NCI-H460 NSCLC cells the concentration response for compound 2, compound 5 and compound 6 were the same. Upon 72 hour exposure of the cells to compound 2, compound 5 and compound 6 the IC₅₀ concentrations were 0.5-0.65 nM and the IC₉₀ concentrations were 1.8-2 nM. To further evaluate the antitumor activity of compound 2, as compared to several approved anticancer agents, the compound was tested in six xenograft models: LOX-IMVI melanoma, DLD-1 and HCT-15 colon, MDA-MB-231 breast, NCI-H292 and NCI-H1299 lung ca. Compound 2 was also compared against two of its metabolites, compound 5 and compound 6, in the HCT-116 colon ca resulting in comparable activity with compound 5. Compound 2 was administered intravenously on a QODx3 schedule for 2 cycles. The tumor growth delay, TGD, (T-C) and increase in lifespan, ILS, (T/C) for each study are listed in the table below.

| | Dose | | TGD | | ILS | |
|---|---|---|---|---|---|---|
| Treatments | (mg/kg/day) | Route/Schedule | (T-C) | | (T/C) | Tumor Line |
| Compound 2 | 1 | IV/QODx3 for 2 cycles | 2 | days | 1.1x | LOX-IMVI |
| Compound 2 | 2 | IV/QODx3 for 2 cycles | 25 | days | 2.8x | |
| Dacarbazine | 90 | IP/QDx5 | 14 | days | 2.0x | |
| Compound 2 | 4 | IV/QODx3 for 2 cycles | 8 | days | 1.2x | DLD-1 |
| CPT-11 | 60 | IV/Q4Dx3 | 5 | days | 1.1x | |
| Compound 2 | 1 | IV/QODx3 for 2 cycles | 14 | days | 1.3x | HCT-15 |
| Compound 2 | 2 | IV/QODx3 for 2 cycles | 35 | days | 1.8x | |
| CPT-11 | 60 | IV/Q4Dx3 | 28 | days | 1.7x | |
| Compound 2 | 1 | IV/QODx3 for 2 cycles | 21 | days | 1.7x | MDA-MB-231 |
| Compound 2 | 1.36 | IV/QODx3 for 2 cycles | >47 | days | >2.3x | |
| Compound 2 | 1.7 | IV/QODx3 for 2 cycles | 35 | days | 2.0x | |
| Docetaxel | 20 | IV/QODx3 | >47 | days | >2.3x | |
| Compound 2 | 1 | IV/QODx3 for 2 cycles | 18 | days | 1.5x | NCI-H292 |
| Compound 2 | 1.36 | IV/QODx3 for 2 cycles | 21 | days | 1.6x | |
| Compound 2 | 1.7 | IV/QODx3 for 2 cycles | 21 | days | 1.6x | |
| Docetaxel | 20 | IV/QODx3 | 39 | days | 2.1x | |
| Compound 2 | 1 | IV/QODx3 for 2 cycles | 20 | days | 1.7x | NCI-H1299 |
| Compound 2 | 1.36 | IV/QODx3 for 2 cycles | 24 | days | 1.8x | |
| Compound 2 | 1.7 | IV/QODx3 for 2 cycles | 34 | days | 2.1x | |
| Docetaxel | 20 | IV/QODx3 | 17 | days | 1.6x | |
| Compound 5 | 4 | IV/QODx3 for 2 cycles | 25 | days | 1.8x | HCT-116 |
| Compound 5 | 6 | IV/QODx3 for 2 cycles | 28 | days | 1.9x | |
| Compound 5 | 8 | IV/QODx3 for 2 cycles | 32 | days | 2.0x | |
| Compound 2 | 1.7 | IV/QODx3 for 2 cycles | 28 | days | 1.9x | |

All of the compound 2 dosages were well tolerated resulting in a maximum body weight loss of ≦20%, except for the high dosages in the HCT-15 and NCI-H292 in which there was a maximum body weight loss of 25.7 and 20.9%, respectively.

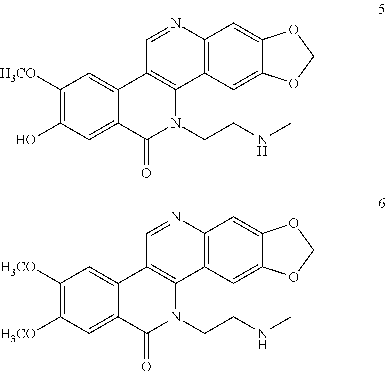

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for treating a cancer selected from colon cancer, non-small cell lung cancer (NSCLC), melanoma, NCI-H292 lung cancer, renal cancer, H1299 lung cancer, colorectal cancer, cervical cancer, breast cancer, and multiple myeloma in a mammal comprising administering to the mammal an effective amount of a compound of formula I: 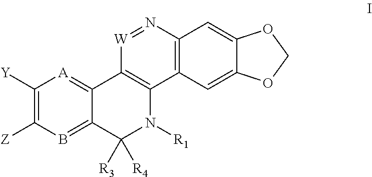
wherein:
A and B are independently N or CH;
W is N or CH;
R₃ and R₄ are each independently H, (C₁-C₆)alkyl, or substituted (C₁-C₆)alkyl, or R₃ and R₄ together are ═O, ═S, ═NH or ═N—R₂;
Y and Z are independently hydroxy, (C₁-C₆)alkoxy, substituted (C₁-C₆)alkoxy, (C₁-C₆)alkanoyloxy, substituted (C₁-C₆) alkanoyloxy, —O—P(═O)(OH)₂, or —O—C(═O)NR_{c}R_{d}; or Y and Z together with the ring carbon atoms to which they are attached form an alkylenedioxy ring with from 5 to 7 ring atoms;
R₁ is a —(C₁-C₆)alkyl substituted with one or more solubilizing groups R_{z};
R₂ is (C₁-C₆)alkyl or substituted (C₁-C₆)alkyl; and
R_{c} and R_{d} are each independently (C₁-C₆) alkyl or substituted (C₁-C₆) alkyl; or R_{c} and R_{d} together with the nitrogen to which they are attached form a N′-{(C₁-C₆)alkyl}piperazino, pyrrolidino, or piperidino ring, which ring can optionally be substituted with one or more aryl, heteroaryl, or heterocycle;
or a pharmaceutically acceptable salt or prodrug thereof.

2. The method of claim 1 wherein A is CH.

3. The method of claim 1 wherein B is CH.

4. The method of any claim 1 wherein Y is —OCH₃.

5. The method of claim 1 wherein Z is OCH₃.

6. The method of claim 1 wherein R₁ is a (C₁-C₆)alkyl substituted with one or more NRₐR_{b} groups.

7. The method of claim 1 wherein R₃ and R₄ together are ═O.

8. The method of claim 1 wherein W is CH.

9. The method of claim 1 wherein the compound is 11,12-dihydro-2,3-dimethoxy-8,9-methylenedioxy-11-{2-(dimethylamino)ethyl}-5,6,11-triazachrysen-12-one, or a pharmaceutically acceptable salt or prodrug thereof.

10. The method of claim 1 wherein the compound of formula I is a compound of formula VIII: 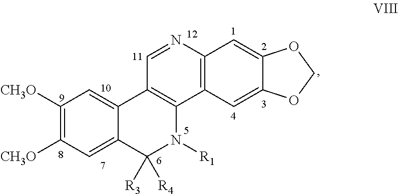
or a pharmaceutically acceptable salt or prodrug thereof.

11. The method of claim 1 wherein the compound of formula I is 8,9-dimethoxy-2,3-methylenedioxy-5-[2-(N,N-dimethylamino)ethyl]-5H-dibenzo[c,h]1,6-naphthyridin-6-one; 8,9-dimethoxy-2,3-methylenedioxy-5-[2-(N,N-diethylamino)ethyl]-5H-dibenzo[c,h]1,6-naphthyridin-6-one; or 8,9-dimethoxy-2,3-methylenedioxy-5-[2-(N-methylamino)ethyl]-5H-dibenzo[c,h]1,6-naphthyridin-6-one; or a pharmaceutically acceptable salt or prodrug thereof.

12. The method of claim 1 wherein the compound of formula I is:
11,12-dihydro-2,3-dimethoxy-8,9-methylenedioxy-11-[2-(dimethylamino)ethyl]-5,6,11-triazachrysen-12-one;
2,3-Dimethoxy-8,9-methylenedioxy-11-[(2-diethylamino)ethyl]-11H-5,6,11-triaza-chrysen-12-one;
2,3-Dimethoxy-8,9-methylenedioxy-11-[(2-dimethylamino)-1-methylethyl]-11H-5,6,11-triaza-chrysen-12-one;
2,3-Dimethoxy-8,9-methylenedioxy-11-(2-tetrahydofuranyl)methyl-11H-5,6,11-triazachrysen-12-one;
2,3-Dimethoxy-8,9-methylenedioxy-11-[2-(pyrro din-l-yl)ethyl]-11H-5,6,11-triaza-chrysen-12-one;
2,3-Dimethoxy-8,9-methylenedioxy-11-[2-(piperidin-l-yl)ethyl]-11H-5,6, 11-triaza-chrysen-12-one;
8,9-Dimethoxy-2,3-methylenedioxy-5-[2-(N,N-dimethylamino)ethyl]-5H-dibenzo[c,h]1,6-naphthyridin-6-one;
8,9-Dimethoxy-2,3-methylenedioxy-5-[2-(N,N-dimethylamino)-1-methylethyl]-5H-dibenzo[c,h]1,6-naphthyridin-6-one;
8,9-Dimethoxy-2,3-methylenedioxy-5-[2-(pyrrolidin-1-yl)ethyl]-5H-dibenzo[c,h]1,6-naphthyridin-6-one;
8,9-Dimethoxy-2,3-methylenedioxy-5-[2-(4-methylpiperazin-1-yl)ethyl]-5H-dibenzo[c,h]1,6-naphthyridin-6-one;
8,9-Dimethoxy-2,3-methylenedioxy-5-[3-(N,N-dimethylamino)propyl]-5H-dibenzo[c,h]1,6-naphthyridin-6-one);
8,9-Dimethoxy-2,3-methylenedioxy-5-(2-tetrahydofuranylmethyl-5H-dibenzo[c,h]1,6-naphthyridin-6-one;
8,9-Dimethoxy-2,3-methylenedioxy-5-[2-(hydroxy)ethyl]-5H-dibenzo[c,h]1,6-naphthyridin-6-one;
8,9-Dimethoxy-2,3-methylenedioxy-5-[2-(2-hydroxyethoxy)ethyl]-5H-dibenzo[c,h]1,6-naphthyridin-6-one;
8,9-Dimethoxy-2,3-methylenedioxy-5-[2-N,N-dimethylamino-1-(hydroxymethyl)ethyl]-5H-dibenzo[c,h]1,6-naphthyridin-6-one;
8,9-Dimethoxy-2,3-methylenedioxy-5-[2,3-dihydroxy)propyl]-5H-dibenzo[c,h]1,6-naphthyridin-6-one;
8,9-Dimethoxy-2,3-methylenedioxy-5-[2-(N,N-dimethylamino)ethyl]-5,6-dihydro-dibenzo[c,h]1,6-naphthyridine;
8,9-Dimethoxy-2,3-methylenedioxy-5-[2-(N,N-dimethylamino)-1-methylethyl]-5,6-dihydro-dibenzo[c,h]1,6-naphthyridine;
8,9-Dimethoxy-2,3-methylenedioxy-5-[2-(N,N-diethylamino)ethyl]-5H-dibenzo[c,h]1,6-naphthyridin-6-one;
2,3-dimethoxy-8,9-methylenedioxy-11-[2-(4-methylpiperazin-1-yl)ethyl]-11H-5,6,11-triazachrysen-12-one;
8,9-dimethoxy-2,3-methylenedioxy-5-(2-piperidinoethyl)-5H-dibenzo[c,h]1,6-naphthyridin-6-one;
8,9-dimethoxy-2,3-methylenedioxy-5-[2-(4-benzylpiperazin-1-yl)ethyl]-5H-dibenzo[c,h]1,6-naphthyridin-6-one;
8,9-dimethoxy-2,3-methylenedioxy-5-formylmethyl-5H-dibenzo[c,h]1,6-naphthyridin-6-one; or
8,9-dimethoxy-2,3-methylenedioxy-5-[2-(N-methylamino)ethyl]-5H-dibenzo[c,h]1,6-naphthyridin-6-one;
or a pharmaceutically acceptable salt or prodrug thereof.

13. The method of claim 1 wherein the cancer is colon cancer.

14. The method of claim 1 wherein the cancer is multiple myeloma.

15. The method of claim 1, wherein the compound is 8,9-dimethoxy-2,3-methylenedioxy-5-[2-(N-methylamino)ethyl]-5H-dibenzo[c,h]1,6-naphthyridin-6-one; or a pharmaceutically acceptable salt or prodrug thereof.

16. The method of claim 1, wherein the compound is 8,9-dimethoxy-2,3-methylenedioxy-5-[2-(N-methylamino)ethyl]-5H-dibenzo[c,h]1,6-naphthyridin-6-one.

17. The method of claim 1, wherein the compound is a citrate salt of 8,9-dimethoxy-2,3-methylenedioxy-5-[2-(N-methylamino)ethyl]-5H-dibenzo[c,h]1,6-naphthyridin-6-one.

18-44. (canceled)
